(19) United States
(12) Patent Application Publication
(10) Pub. No.: US 2011/0286958 A1
(43) Pub. Date: Nov. 24, 2011

(54) HPMA POLYMER PLATINUM CHELATES

(75) Inventors: Paul Sood, Dallas, TX (US); N. Rao Ummaneni, Dallas, TX (US); Bruce Thurmond, Dallas, TX (US); Ryszard Zarzycki, Dallas, TX (US)

(73) Assignee: Access Pharmaceuticals, Inc.

(21) Appl. No.: 13/108,872

(22) Filed: May 16, 2011

Related U.S. Application Data
(60) Provisional application No. 61/345,517, filed on May 17, 2010.

(51) Int. Cl. A61K 31/785; C07K 16/00; C07K 14/00; C07J 1/00; C07K 14/655; C07K 14/42; C07D 475/04; C07H 23/00; C07D 495/04; C09B 47/00; C07H 1/00; A61P 35/00; C08G 63/91; C08G 77/58; C07C 229/26

(52) U.S. Cl. 424/78.37; 560/169; 530/388.1; 530/389.1; 530/300; 530/350; 552/502; 530/327; 530/396; 544/258; 536/26.4; 548/303.7; 540/121; 554/107; 536/123.1; 525/54.1; 525/54.2; 525/389

(57) ABSTRACT

Polymeric platinum amidomalonate complexes, where the platinum is in +2 or +4 oxidation state, and where the complexes optionally contain tumor seeking groups, are useful in the treatment of cancer.

### CROSS-REFERENCE TO RELATED APPLICATIONS

This application claims the benefit under 35 U.S.C. §119(e) of U.S. Provisional Application No. 61/345,517, filed on May 17, 2010, the contents of which are hereby incorporated by reference in their entirety into the present disclosure.

### FIELD OF THE INVENTION

The present invention relates to platinum complexes bound to hydroxypropylmethacrylamide (HPMA) copolymers, useful for the treatment of cancer.

### BACKGROUND OF THE INVENTION

Since the advent of cisplatin (I) in the late 1960s, platinum complexes have become a mainstay in the practitioner's arsenal of anti-tumor chemotherapeutics. They find use, either alone or in combination with other chemotherapeutic agents, against virtually all solid tumor cancers. All current clinical platinum complex chemotherapeutics share the generic structure of cisplatin; i.e., Pt(L₁)(L₂)(L₃)(L₄), in which L₁ and L₂ represent two stable cis-monodentate am(m)ine ligands or, as L₁-L₂, a stable bidentate amine ligand and L₃ and L₄ represent two monodentate anionic leaving-ligands or, as L₃-L₄, a bidentate anionic leaving-ligand. Despite their ability to react with many different biomolecules, the mode of action of the Pt complex drugs is presently accepted as involving hydrolytic loss of the anionic leaving-ligands with concomitant formation of the much more reactive aqua (water) ligand complex, which is capable of reacting with DNA to form intra- and inter-strand cross-links, leading to cell death. The usefulness of cisplatin is limited by its therapeutic index (the ratio of the maximum tolerable dose to minimum effective dose), which tends to be relatively low due to the toxicity of the active aqua species and the rapidity with which it forms in vivo. Among the approaches that have been employed to improve the therapeutic index of cisplatin, two have predominated. The first had been to use anionic leaving-ligands that are more stable to hydrolytic cleavage so that the aqua species does not form until the compound has infiltrated a target tumor. To date, this approach has resulted in four clinical platinum complex compounds that exhibit improved pharmaceutical characteristics compared to cisplatin: carboplatin (II), oxaliplatin (III), nedaplatin (IV) and lobaplatin (V).

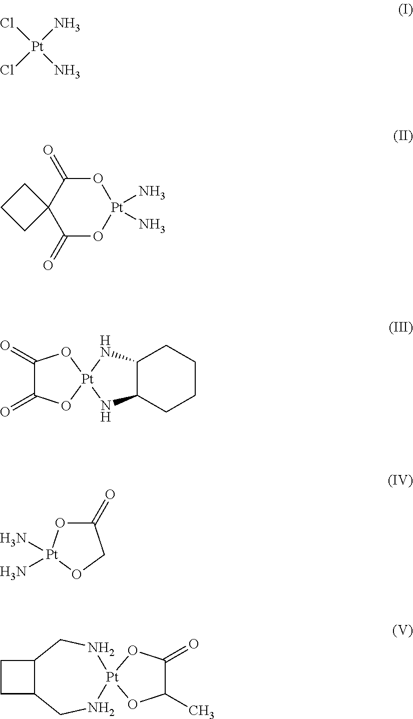

The second approach, often used in conjunction with the first, is targeting; i.e., combining the platinum complex with a compound that preferentially accumulates in tumors so that, once it passively encounters a tumor or a compound that has a specific affinity for a molecule or receptor expressed on the surface of a neoplastic cell but not a healthy cell. Preferential accumulation in tumors can be achieved by using compounds that take advantage of the “enhanced permeability and retention” (EPR) effect operative in tumors.

Briefly, the EPR effect, which was first described with regard to the preferential permeation into, and retention by, tumor tissues of serum proteins, is the result of defective tissue architecture, changes in permeation mediators and impaired lymphatic drainage in tumors. That is, the vascular endothelium of tumors tends to have relatively large gaps in the endothelial cell-cell junctions compared to normal tissue. This permits larger molecular species to permeate the tissue than is the case for healthy tissue. The altered permeation mediators and impaired lymphatic drainage mechanism then assure that the molecules that have penetrated the tumor stay there. The EPR effect has been used in practice to selectively introduce and retain chemotherapeutics in tumors by tethering small molecule drugs to polymers or nanoparticles that are too large to permeate normal tissue but that readily infiltrate tumor tissue.

For example, U.S. Pat. No. 5,965,118, (the '118 patent) assigned to Access Pharmaceuticals, claims a polyacrylamide or polymethacrylamide backbone polymer wherein a portion of the pendant amide groups are linked by a peptide chain to a terminal platinum complex. The remainder of the pendant amide groups are substituted with a water-solubilizing hydroxyalkyl group. The size of the polymer is optimized to be large enough to take advantage of the EPR effect, yet small enough so that any drug remaining in the circulatory system is susceptible to renal elimination. The anionic leaving ligands through which the Pt species are bound to the polymer are bidentate carboxyl or aminoethylamido groups in the '118 patent. The polymer-bound platinum complex is passively transported to the target tumor through the vascular system until it preferentially enters into and is retained in tumors due to the EPR effect where hydrolytic cleavage to an active small molecule aqua species occurs.

U.S. Pat. No. 6,692,734, also assigned to Access Pharmaceuticals, likewise claims polymer-bound platinum complexes, the difference being that the Pt is bound to the polymer by bidentate N,O-amidomalonate ligands rather than bidentate aminoethylamide or bidentate carboxyl ligands. U.S. Pat. No. 7,166,733, also assigned to Access Pharmaceuticals, claims product-by process for polymer-bound platinum complexes, particularly control of N,O-versus O,O-binding of platinum to the chelating group attached to the polymer. U.S. Patent Application 2005/0038109, also assigned to Access Pharmaceuticals, claims several additional platinum constructs optionally bound to polymers for effective delivery of platinum to tumors. In addition to the research and development work which is the subject matter of the aforementioned series of patents, other researchers have examined the possibility of using polymers for the enhancement of delivery of platinum to tumors. For example, U.S. Pat. Nos. 4,551,502 and 4,278,660 claims square planar platinum complexes linked to a water-soluble polymer having at least one amidocarbonylic unit for binding the metal. U.S. Pat. No. 4,793,986 claims complexes of platinum with certain polysaccharide carriers as antitumor agents. U.S. Pat. No. 5,252,713 describes polypeptides as carriers of platinum (or other anticancer compounds), optionally attached to monoclonal antibodies for additional tumor targeting.

The use of polymers for targeting of platinum to tumors has also been the subject of recent reviews, for example:
Siegmann-Louda D W, Carraher C E Jr., (2004) Polymeric platinum containing drugs in the treatment of cancer. Macromolecules Containing Metal and Metal-like Elements, Volume 3: Biomedical Applications, pp 119-191, Wiley, New York.
Neuse E W. Synthetic polymers as drug-delivery vehicles in medicine. Met Based Drugs. 2008: 469531;
Haxton K J, Burt H M, Polymeric drug delivery of platinum-based anticancer agents, J Pharm Sci. 2009: 98(7):2299-316;
Fox M E, Szoka F C, Fréchet J M. Soluble polymer carriers for the treatment of cancer: the importance of molecular architecture, Acc Chem. Res. 2009: 42(8):1141-1151;
Maeda H, Bharate G Y, Daruwalla J., Polymeric drugs for efficient tumor-targeted drug delivery based on EPR-effect, Eur J Pharm Biopharm. 2009: 71(3), 409-19; and
Duncan R, Vincent M J, Do HPMA copolymer conjugates have a future as clinically useful nanomedicines? A critical overview of current status and future opportunities, Adv Drug Deliv Rev. 2010: 62(2), 272-282.

In spite of the large amount of research and development effort that has gone into the discovery and progression of polymer-linked platinum drugs, very few have advanced to clinical development. One of the major issues has been compliance with the Regulatory requirements for manufacturing consistency, ensuring that the strength, quality, purity, and potency of polymer-based drugs are well-controlled and meet prescribed specifications from batch-to-batch. This has proven to be particularly challenging when compared with small molecule drugs. Polymer carriers are inherently mixtures given their variability in molecular weight and random distribution of monomer units within the polymer strands. Therefore, it is necessary to ensure that the polymer can be manufactured consistently from batch-to-batch by meeting specifications on parameters that may affect the pharmaceutical and pharmacological properties of the resultant drug substance. Similarly, attachment of the platinum to the polymer must be performed in a manner which provides a consistent product over multiple batches. Additionally, the processes used to manufacture these products under current Good Manufacturing Practices (cGMPs) must be both scalable to commercial levels and capable of providing the polymer-drug product in a cost-effective manner.

Previously, HPMA-polymer platinum conjugates have been made by processes which have worked well in the research environment, but had proven problematic for manufacture at larger scale. These processes involved the initial formation of the polymer, subsequent attachment of linking and chelating groups, followed by attachment of the platinum derivative to yield the final product. Formation of the chelating polymer in two or more steps from the monomers is less than ideal for providing a consistent product. Similarly, attachment of platinum to the chelating polymer through the use of an insoluble resin for absorption of counter ions, as had been previously described and implemented, is also less than ideal for consistency, cost of the resin, scalability and overall yield.

Additionally, the manner in which platinum is attached to the polymer can have an impact on its in vivo release profile. Without adequate controls during the process of platinum attachment to the polymer chain, the platinum release characteristics could vary from batch-to-batch, leading to variability in both efficacy and safety of the product. Additionally, the process must provide a consistent high yield of platinum incorporation in order that the product is commercially viable from a cost perspective.

Furthermore, for delivery of a platinum agent which possesses one or more chiral centers, the stereochemistry and stereochemical purity of the attached platinum species must be controlled. It is well known in the art that the stereochemistry of active species can have a profound impact on efficacy and toxic side-effects. It is therefore essential that the stereochemistry of the platinum species attached to the polymer remains unchanged during the process of addition to the polymer, during storage, and during the preparation of the injectable pharmaceutical. Active platinum species with high stereochemical purity of single stereochemical isomers are preferred.

### SUMMARY OF THE INVENTION

Thus, in one aspect, the present invention relates to a compound having the chemical structure:

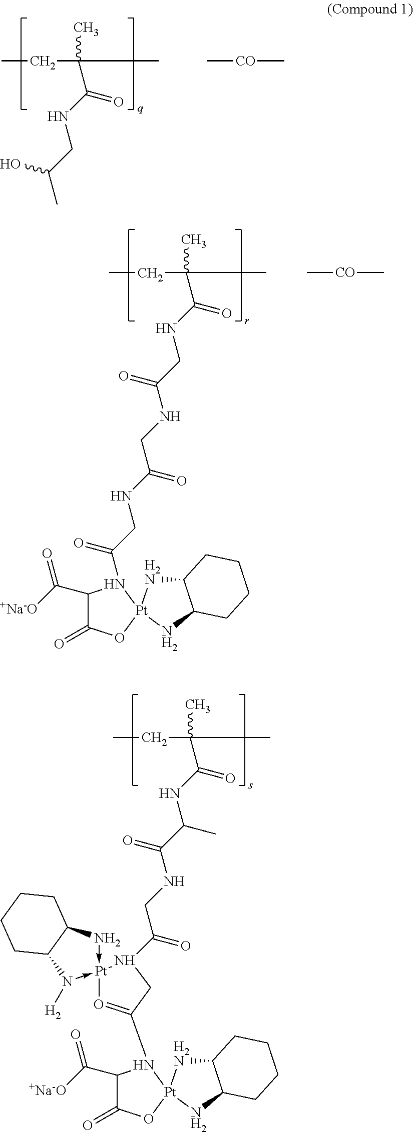

wherein:
‘co’ represents binding of the individual monomer units to form a random copolymer.
q, r, and s represent the molar percentage of the monomer units; such that
q+r+s=100 and
q is ≧50 and q≦95;
r is ≧5 and r≦50;
s is ≧0 and s≦30;
Pt is in a +2 or a +4 oxidation state.

Also provided is a polymeric platinum complex comprising a polymer of formula 1a:

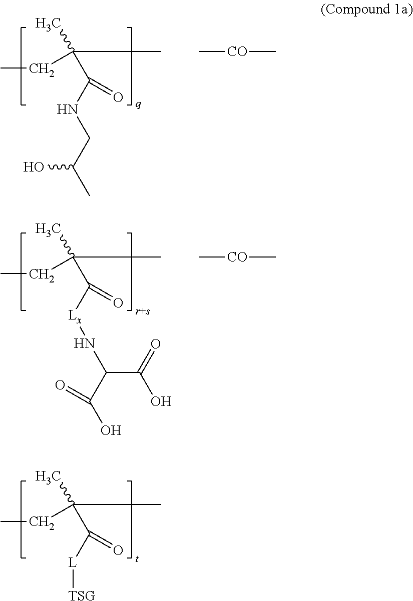

or a compound of the formula:

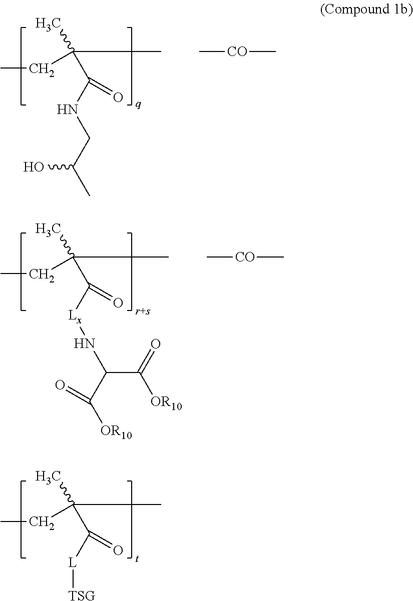

wherein Compound 1b is a polymer to which a platinum cytotoxic agent is chelated, and for each of Compounds 1a and 1b,
‘co’ represents binding of the individual monomer units to form a random copolymer.
q, r+s, and t represent the molar percentage of the monomer units; q+r+s+t=100;
q is ≧50 and ≦95;
r+s is ≧5 and ≦50;
t is ≧0 and ≦30;
provided however that, t is 0 only when Lₓ is a bond;
TSG is a tumor seeking group that is capable of targeting cancerous tumor selected from the group of a monoclonal antibody, an antibody fragment, a peptide comprising 2-50 amino acids, a protein, a steroid, a somatostatin analog, a lectin, a folic acid or its derivatives and analogs, vitamin B12, biotin, porphyrin, an essential fatty acid, a bioreductive molecule and a polyanionic polysaccharide;
L and Lx are independently a covalent bond, or an amino acid, or a peptide comprising 2-50 amino acids, or a straight or branched C₁-C₁₀ alkylene chain; or a combination of these components;
or a salt of each of the above.

An aspect of this invention is a compound (used here interchangeably with the term “a polymeric platinum complex”) as described herein in which the DACH (diaminocyclohexane) group is in the R,R′-stereochemical configuration.

An aspect of this invention is a compound as described herein in which the DACH (diaminocyclohexane) group is in the R,R′-stereochemical configuration possessing a stereochemical purity >95%.

An aspect of this invention is a compound as described herein in which the DACH (diaminocyclohexane) group is in the S,S′-stereochemical configuration.

An aspect of this invention is a compound as described herein in which the DACH (diaminocyclohexane) group is in the S,S′-stereochemical configuration possessing a stereochemical purity >95%.

An aspect of this invention is a compound as described herein in which the DACH (diaminocyclohexane) group is in both the R,R′- and the S,S′-stereochemical configuration; that is, a mixture of the two stereochemical forms.

An aspect of this invention is a compound as described herein in which the DACH (diaminocyclohexane) group is in both the R,R′- and the S,S′-stereochemical configuration; that is, a mixture of the two stereochemical forms, wherein the ratio of the two stereoisomers is in a narrow range with the proportion of R,R′-<=95% at one extreme and R,R′->=5% at the other extreme.

An aspect of this invention is a compound as described herein in which all or some Pt atoms are in the +2 oxidation state.

An aspect of this invention is a compound as described herein in which all or some Pt atoms are in the +4 oxidation state.

An aspect of this invention is a compound in which all or some Pt atoms are attached to the two oxygen atoms of the malonate groups;

An aspect of this invention is a compound as described herein in which all or some Pt atoms are attached to oxygen and nitrogen atoms of the peptide spacer group other than the points of attachment shown in the above structure.

A further aspect of this invention are processes for the attachment of platinum atoms to the polymer which provide Compound 1, 1a or 1b in a consistent manner and with a high yield with respect to platinum employed.

A further aspect of this invention wherein Compound 1, 1a or 1b is purified by a process which separates the polymer from low molecular weight materials.

A further aspect of this invention wherein Compound 2 is purified by a process which separates the polymer from low molecular weight materials.

A further aspect of this invention is in the use of Compound 1, 1a or 1b for the treatment of cancer.

A further aspect of this invention is in the use of Compound 1, 1a or 1b for the treatment of solid tumor cancers.

A further aspect of this invention is in the use of Compound 1, 1a or 1b for the treatment of ovarian cancer.

A further aspect of this invention is in the use of Compound 1, 1a or 1b for the treatment of gastric cancer.

A further aspect of this invention is in the use of Compound 1, 1a or 1b for the treatment of lung cancer.

A further aspect of this invention is in the use of Compound 1, 1a or 1b for the treatment of melanoma.

A further aspect of this invention is in the use of Compound 1, 1a or 1b in combination with one or more other anticancer compounds for the treatment of cancer.

A further aspect of this invention is in the use of Compound 1, 1a or 1b in combination with one or more other chemotherapeutic for the treatment of cancer.

A further aspect of this invention is in the use of Compound 1, 1a or 1b in combination with paclitaxel for the treatment of cancer.

A further aspect of this invention is in the use of Compound 1, 1a or 1b in combination with gemcitabine for the treatment of cancer.

A further aspect of this invention is in the use of Compound 1, 1a or 1b in combination with 5-fluorouracil and leucovorin for the treatment of cancer.

A further aspect of this invention is in the use of Compound 1, 1a or 1b in combination with vinorelbine for the treatment of cancer.

A further aspect of this invention is in the use of Compound 1, 1a or 1b in combination with paclitaxel for the treatment of cancer wherein Compound 1, 1a or 1b and paclitaxel are administered intravenously to the patient once every three weeks.

A further aspect of this invention is in the use of Compound 1, 1a or 1b in combination with paclitaxel for the treatment of cancer wherein compound 1 and paclitaxel are administered intravenously to the patient once every two weeks.

A further aspect of this invention is in the use of Compound 1, 1a or 1b in combination with paclitaxel for the treatment of cancer wherein compound 1 is administered intravenously to the patient once every three weeks and paclitaxel is administered intravenously to the patient once every two weeks.

A further aspect of this invention is in the use of Compound 1, 1a or 1b in combination with paclitaxel for the treatment of cancer wherein Compound 1, 1a or 1b is administered intravenously to the patient once every two weeks and paclitaxel is administered intravenously to the patient once every three weeks.

A further aspect of this invention is in the preparation of a pharmaceutical product comprising, consisting essentially of, or consisting of Compound 1, 1a or 1b optionally formulated and intimately mixed with one or more pharmaceutically-acceptable excipients provided as a lyophilized powder in a sterile sealed vial wherein the vial contents are dissolved in a vehicle suitable for administration to a patient just prior to use.

A further aspect of this invention is in the preparation of a pharmaceutical product comprising, consisting essentially of, or consisting of Compound 1, 1a or 1b optionally formulated and with one or more pharmaceutically-acceptable excipients dissolved in a vehicle suitable for administration to a patient provided in a sterile sealed vial.

In a further aspect, the present invention also relates to a compound having the chemical structure:

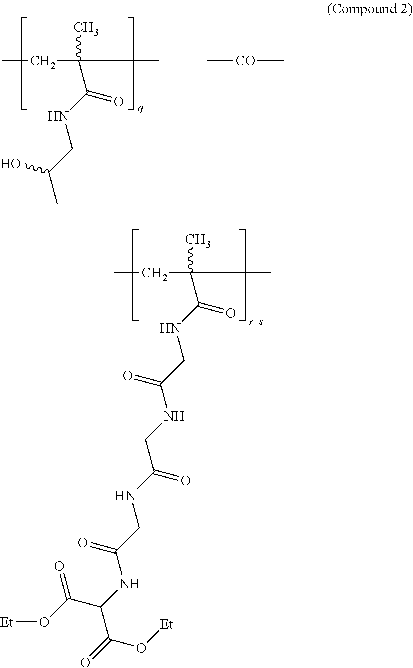

wherein:
‘co’ represents binding of the individual monomer units to form a random copolymer.
q and r+s represent the molar percentage of the monomer units; such that
q+r+s=100 and
q is >50 and q<95;
r+s is >5 and r<50;
and wherein Compound 2 is used to manufacture Compound 1 using the processes disclosed herein such that Compound 1 is made from Compound 2 in a consistent manner at scales ranging from milligrams to scales ≧100 kilograms.

An aspect of this invention is a process whereby DACH platinum is attached to the saponified form of Compound 2 to provide a product with consistent loading of DACH platinum.

An aspect of this invention is a process in which the addition of DACH platinum to the saponified form of compound is performed maintaining tight control of rates of addition, pH, and temperature.

An aspect of this invention is a compound in which all or some Pt atoms are attached to oxygen and nitrogen atoms of the peptide spacer group other than the points of attachment shown in the structure, Compound 1.

An aspect of this invention is a compound in which Pt atoms are bound to the amidomalonato chelating group of Compounds 1a and 1b.

An aspect of this invention is a compound in which Pt atoms can be bound to the amidomalonato chelating group of Compounds 1a and 1b and may also be attached to oxygen and nitrogen atoms of the peptide spacer group.

In a further aspect, the present invention also relates to compounds having the following chemical structures:

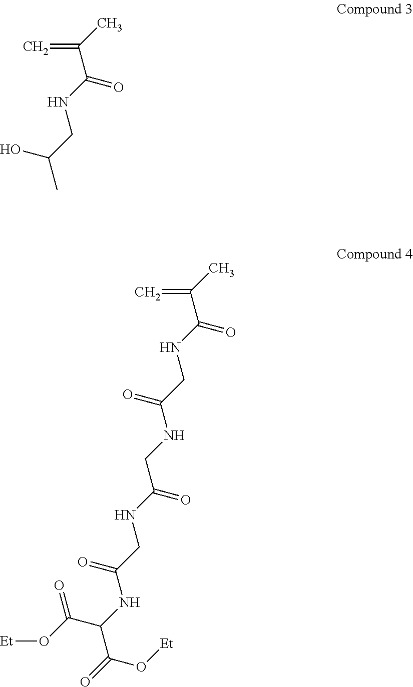

While Compound 3 (N-methacroyl-1-amino-2-propanol) is well known in the art as a reagent (monomer) for the formation of HPMA polymers and copolymers, Compound 4 is novel. Copolymerization of Compounds 3 and 4 results in the formation of Compound 2; a process not previously described and which has surprising benefits over previously published processes to prepare Compound 2.

An aspect of this is invention is Compound 4 and the use of compound form in a chemical process, wherein Compound 4 is a reagent (monomer) that can be reacted with Compound 3 to form the Compound 2 utilizing a suitable solvent and suitable free-radical initiation agent. Compounds obtainable by this process are also provided herein.

An aspect of this invention is the controlled free-radical polymerization of Compounds 3 and 4 to produce Compound 2 in a highly reproducible manner.

An aspect of this invention is the controlled free-radical polymerization of Compounds 3 and 4 to produce Compound 2 in a highly reproducible manner in gram quantities.

An aspect of this invention is the controlled free-radical polymerization of Compounds 3 and 4 to produce Compound 2 in a highly reproducible manner in multikilogram quantities.

An aspect of this invention is the controlled free-radical polymerization of Compounds 3 and 4 to produce Compound 2 in a highly reproducible manner through temperature control and control of heating and cooling rates.

A further aspect of this invention wherein Compound 2 is made by free-radical polymerization of Compound 3 with a dialkyl ester of N-methacroyl-triglycyl-aminomalonate in a suitable solvent and suitable free-radical initiation agent wherein the term ‘alkyl’ refers to a hydrocarbon as later defined.

A further aspect of this invention wherein a copolymer is made by free-radical polymerization of Compound 3 with a dialkyl ester of N-methacroyl-linker-aminomalonate in a suitable solvent and suitable free-radical initiation agent wherein the term ‘linker’ refers to a functional group as later defined.

A further aspect of this invention wherein a copolymer is made by free-radical polymerization of Compound 3 with a compound being an N-methacroyl-linker-bound to a suitable protected or unprotected chelating agent in a suitable solvent and suitable free-radical initiation agent wherein the terms ‘linker’ and ‘chelating agent’ refer to functional groups as later defined.

In a further aspect, the present invention also relates to a compound having the chemical structure:

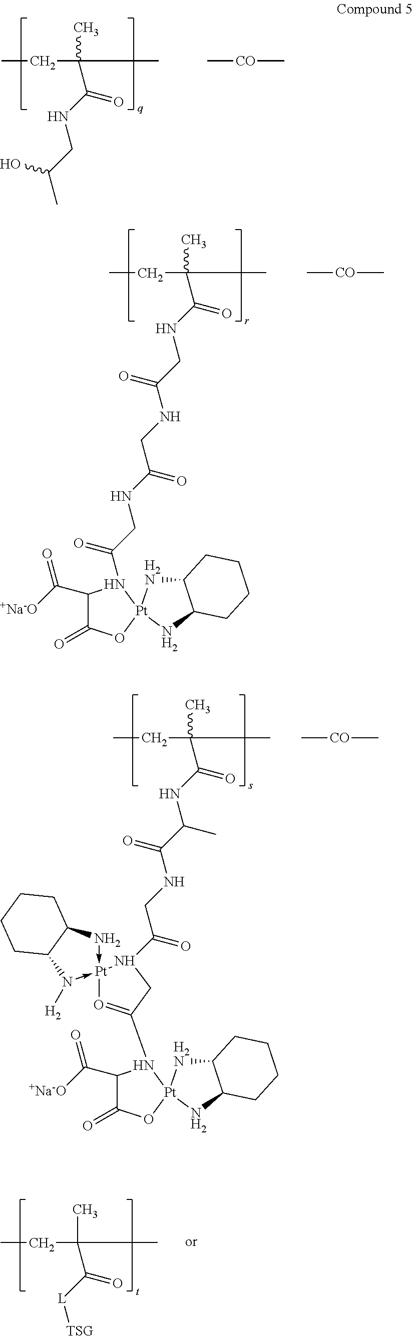

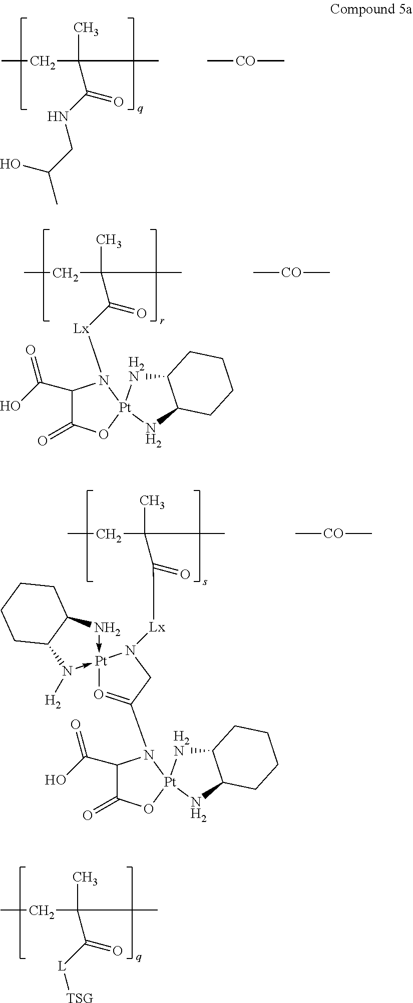

wherein:
‘co’ represents binding of the individual monomer units to form a random copolymer.
L is a linker group which can be a covalent bond, or an amino acid, or a peptide, or a straight or branched alkyl chain; or a combination of these components, or as later defined;
Lx can be the same or different and are independently a covalent bond, or an amino acid, or a peptide comprising 2-50 amino acids, or a straight or branched C₁-C₁₀ alkylene chain; or a combination of these components;
q, r, s and t represent the molar percentage of the monomer units; such that
q+r+s+t=100 and
q is ≧50 and q≦95;
r is ≧5 and r≦50;
s is ≧0 and s≦30;
t is ≧0 and t≦30;
Pt is in a +2 or a +4 oxidation state; and
TSG is a tumor seeking group that is capable of targeting cancerous tumor, non-limiting examples of which are selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharides.

A further aspect of this invention wherein Compound 5 or 5a is purified by a process which separates the polymer from low molecular weight materials.

A further aspect of this invention is in the use of Compound 5 or 5a for the treatment of cancer.

A further aspect of this invention is in the use of Compound 5 or 5a for the treatment of solid tumor cancers.

A further aspect of this invention is in the use of Compound 5 or 5a for the treatment of ovarian cancer.

A further aspect of this invention is in the use of Compound 5 or 5a for the treatment of gastric cancer.

A further aspect of this invention is in the use of Compound 5 or 5a for the treatment of lung cancer.

A further aspect of this invention is in the use of Compound 5 or 5a for the treatment of melanoma.

A further aspect of this invention is in the use of Compound 5 or 5a in combination with one or more other anticancer compounds for the treatment of cancer.

A further aspect of this invention is in the use of Compound 5 or 5a in combination with one or more other chemotherapeutic for the treatment of cancer.

A further aspect of this invention is in the use of Compound 5 or 5a in combination with paclitaxel for the treatment of cancer.

A further aspect of this invention is in the use of Compound 5 or 5a in combination with gemcitabine for the treatment of cancer.

A further aspect of this invention is in the use of Compound 5 or 5a in combination with 5-fluorouracil and leucovorin for the treatment of cancer.

A further aspect of this invention is in the use of Compound 5 or 5a in combination with vinorelbine for the treatment of cancer.

A further aspect of this invention is in the use of Compound 5 or 5a in combination with paclitaxel for the treatment of cancer wherein Compound 5 or 5a and paclitaxel are administered intravenously to the patient once every three weeks.

A further aspect of this invention is in the use of Compound 5 or 5a in combination with paclitaxel for the treatment of cancer wherein Compound 5 or 5a and paclitaxel are administered intravenously to the patient once every two weeks.

A further aspect of this invention is in the use of Compound 5 or 5a in combination with paclitaxel for the treatment of cancer wherein Compound 5 or 5a is administered intravenously to the patient once every three weeks and paclitaxel is administered intravenously to the patient once every two weeks.

A further aspect of this invention is in the use of Compound 5 or 5a in combination with paclitaxel for the treatment of cancer wherein Compound 5 or 5a is administered intravenously to the patient once every two weeks and paclitaxel is administered intravenously to the patient once every three weeks.

A further aspect of this invention is in the preparation of a pharmaceutical product and/or a kit comprising, consisting essentially of, or consisting of Compound 5 or 5a optionally formulated and intimately mixed with one or more pharmaceutically-acceptable excipients provided as a lyophilized powder in a sterile sealed vial wherein the vial contents are dissolved in a vehicle suitable for administration to a patient just prior to use.

A further aspect of this invention is in the preparation of a pharmaceutical product and/or a kit comprising, consisting essentially of, or consisting of Compound 5 or 5a optionally formulated and with one or more pharmaceutically-acceptable excipients dissolved in a vehicle suitable for administration to a patient provided in a sterile sealed vial.

An aspect of this invention is a compound as described herein wherein the DACH (diaminocyclohexane) group is in the R,R′-stereochemical configuration.

An aspect of this invention is a compound as described herein wherein the DACH (diaminocyclohexane) group is in the R,R′-stereochemical configuration possessing a stereochemical purity >95%.

An aspect of this invention is a compound as described herein wherein the DACH (diaminocyclohexane) group is in the S,S′-stereochemical configuration.

An aspect of this invention is a compound as described herein wherein the DACH (diaminocyclohexane) group is in the S,S′-stereochemical configuration possessing a stereochemical purity >95%.

An aspect of this invention is a compound as described herein wherein the DACH (diaminocyclohexane) group is in both the R,R′- and the S,S′-stereochemical configuration; that is, a mixture of the two stereochemical forms.

An aspect of this invention is a compound as described herein wherein the DACH (diaminocyclohexane) group is in both the R,R′- and the S,S′-stereochemical configuration; that is, a mixture of the two stereochemical forms, wherein the ratio of the two stereoisomers is in a narrow range with the proportion of R,R′-<=95% at one extreme and R,R′->=5% at the other extreme.

An aspect of this invention is a compound as described herein wherein in which all or some Pt atoms are in the +2 oxidation state.

An aspect of this invention is a compound as described herein wherein in which all or some Pt atoms are in the +4 oxidation state.

An aspect of this invention is a compound as described herein wherein in which all or some Pt atoms are attached to the two oxygen atoms of the malonate groups;

An aspect of this invention is a compound in which all or some Pt atoms are attached to oxygen and nitrogen atoms of the peptide spacer group other than the points of attachment shown in the above structure.

A further aspect of this invention are processes for the attachment of platinum atoms to the polymer which provide Compound 1, 1a or 1b or Compound 5 or 5a in a consistent manner and with a high yield with respect to platinum employed.

A further aspect of the present invention provides a polymeric complex comprising, consisting essentially of, or consisting of a compound of Formula 6

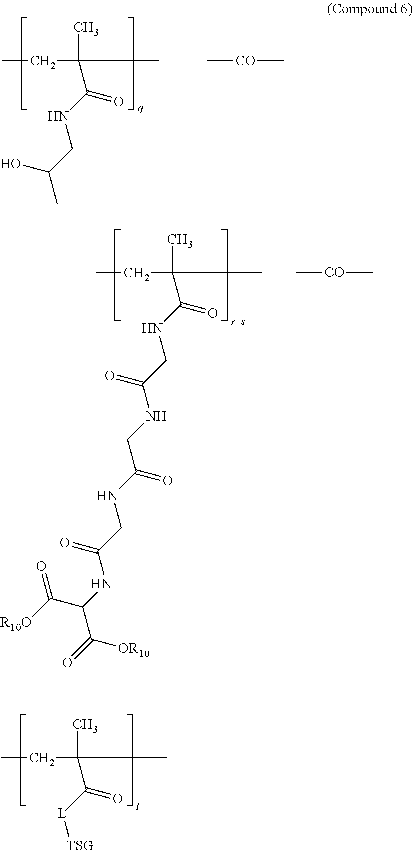

wherein
‘co’ represents binding of the individual monomer units to form a random copolymer;
L is a linker group which can be a covalent bond, or an amino acid, or a peptide, or a straight or branched alkyl chain; or a combination of these components, or as later defined;
TSG is a tumor seeking group that is capable of targeting cancerous tumor, non-limiting examples of which are selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharides;
q, r+s, and t represent the molar percentage of the monomer units; such that
q+r+s+t=100 and
q is ≧50 and q≦95;
r+s is ≧5 and r+s≦50;
t is ≧0 and t≦30;
R₁₀ is alkyl;
and wherein Compound 5 or 5a is used to manufacture a platinum compound similar to that shown as Compound 1, 1a or 1b wherein platinum is bound through two coordination bonds to the amidomalonate chelating group and/on the peptide linker, and using the processes disclosed herein such that the platinum compound is made from Compound 5 or 5a in a consistent manner at scales ranging from milligrams to scales ≧100 kilograms;
and wherein Compound 6 is made by copolymerization of Compound 3, Compound 4, and a monomer containing the targeting group, wherein the targeting group is either unprotected or is protected using a protecting group as known in the art to prevent chemical modification of the targeting group during the polymerization process.

In a further aspect, the present invention also relates to compounds having the following chemical structures:

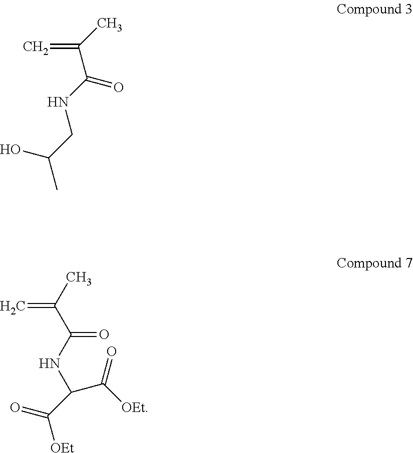

While Compound 3 (N-methacroyl-1-amino-2-propanol) is well known in the art as a reagent (monomer) for the formation of HPMA polymers and copolymers, to the best of Applicant's knowledge, Compound 7 is novel. Copolymerization of Compounds 3 and 7 results in the formation of novel Compound 8:

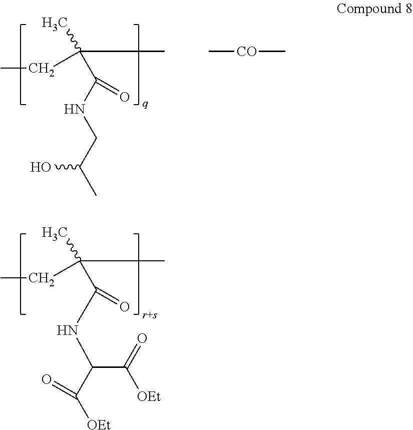

wherein:
‘co’ represents binding of the individual monomer units to form a random copolymer.
q and r+s represent the molar percentage of the monomer units; such that
q+r+s=100 and
q is ≧50 and q≦95
r+s is ≧5 and r≦50;
and wherein Compound 8 is used to manufacture Compound 9 using the processes disclosed herein such that Compound 9 is made from Compound 8 in a consistent manner at scales ranging from milligrams to scales ≧100 kilograms.

An aspect of this is invention is Compound 7, which is a reagent (monomer) that can be reacted with Compound 3 to form the Compound 8 utilizing a suitable solvent and suitable free-radical initiation agent.

An aspect of this invention is the controlled free-radical polymerization of Compounds 3 and 7 to produce Compound 8 in a highly reproducible manner.

An aspect of this invention is the controlled free-radical polymerization of Compounds 3 and 7 to produce Compound 8 in a highly reproducible manner in gram quantities.

An aspect of this invention is the controlled free-radical polymerization of Compounds 3 and 7 to produce Compound 8 in a highly reproducible manner in multi kilogram quantities.

An aspect of this invention is the controlled free-radical polymerization of Compounds 3 and 7 to produce Compound 8 in a highly reproducible manner through temperature control and control of heating and cooling rates.

In a further aspect, the present invention relates to a compound having the chemical structure:

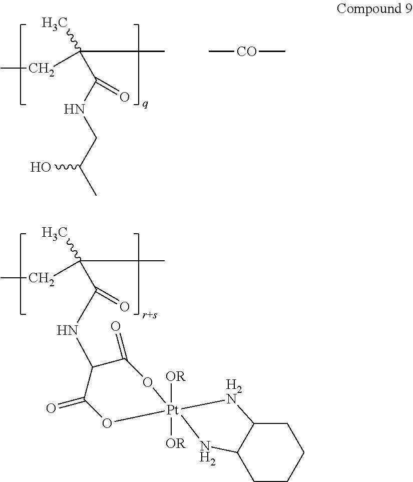

wherein:
‘co’ represents binding of the individual monomer units to form a random copolymer.
q, r, and s represent the molar percentage of the monomer units; such that
q+r+s=100 and
q is ≧50 and q≦95;
r is ≧5 and r≦50;
s is ≧0 and s≦30;
Pt is in a +2 or a +4 oxidation state.

An aspect of this invention is a compound as described herein in which the DACH (diaminocyclohexane) group is in the R,R′-stereochemical configuration.

An aspect of this invention is a compound as described herein in which the DACH (diaminocyclohexane) group is in the R,R′-stereochemical configuration possessing a stereochemical purity >95%.

An aspect of this invention is a compound as described herein in which the DACH (diaminocyclohexane) group is in the S,S′-stereochemical configuration.

An aspect of this invention is a compound as described herein in which the DACH (diaminocyclohexane) group is in the S,S′-stereochemical configuration possessing a stereochemical purity >95%.

An aspect of this invention is a compound as described herein in which the DACH (diaminocyclohexane) group is in both the R,R′- and the S,S′-stereochemical configuration; that is, a mixture of the two stereochemical forms.

An aspect of this invention is a compound as described herein in which the DACH (diaminocyclohexane) group is in both the R,R′- and the S,S′-stereochemical configuration; that is, a mixture of the two stereochemical forms, wherein the ratio of the two stereoisomers is in a narrow range with the proportion of R,R′-<=95% at one extreme and R,R′->=5% at the other extreme.

An aspect of this invention is a compound as described herein in which all or some Pt atoms are in the +2 oxidation state.

An aspect of this invention is a compound as described herein in which all or some Pt atoms are in the +4 oxidation state.

An aspect of this invention is a compound as described herein in which all or some Pt atoms are attached to the two oxygen atoms of the malonate groups.

A further aspect of this invention are processes for the attachment of platinum atoms to the polymer which provide Compound 9 in a consistent manner and with a high yield with respect to platinum employed.

In a further aspect, the present invention also relates to a compound having the chemical structure:

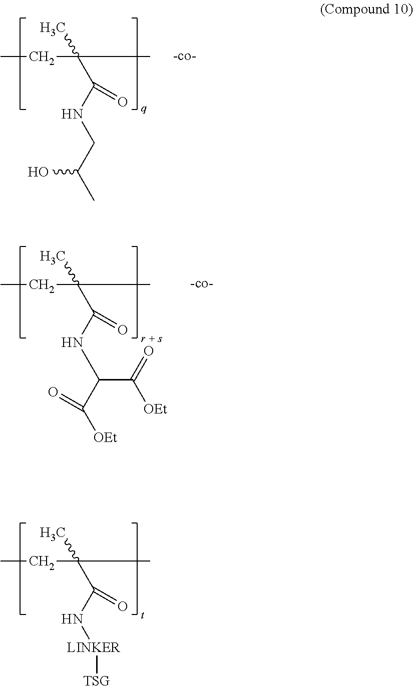

wherein:
‘co’ represents binding of the individual monomer units to form a random copolymer.
q, r+s, and t represent the molar percentage of the monomer units; such that
q+r+s+t=100 and
q is ≧50 and q≦95;
r+s is ≧5 and r+s≦50;
t is ≧0 and t≦30;
TSG is a tumor seeking group that is capable of targeting cancerous tumor, non-limiting example of which are selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharides;
L is a linker group which can be a covalent bond, or an amino acid, or a peptide, or a straight or branched alkyl chain; or a combination of these components, or as later defined;
wherein the targeting group is covalently bound to the linker and the linker is covalently bound to the polymer and the targeting group and the linker are as defined herein.

In a further aspect, the present invention also relates to a compound having the chemical structure:

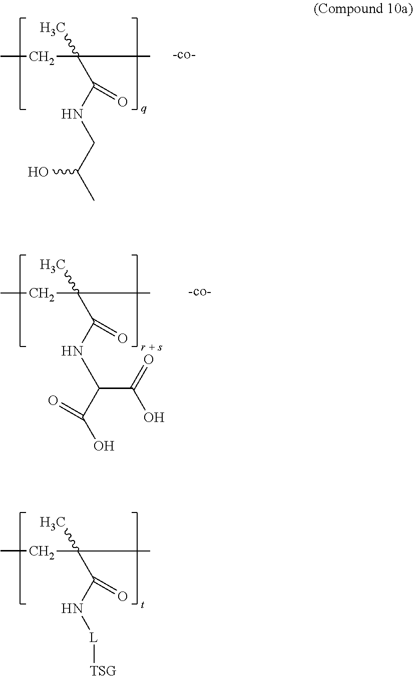

wherein:
‘co’ represents binding of the individual monomer units to form a random copolymer.
q, r+s, and t represent the molar percentage of the monomer units; such that
q+r+s+t=100 and
q is ≧50 and q≦95
r+s is ≧5 and r+s≦50;
t is ≧0 and t≦30;
TSG is a tumor seeking group that is capable of targeting cancerous tumor, non-limiting examples of which are selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharides;
L is a linker group which can be a covalent bond, or an amino acid, or a peptide, or a straight or branched alkyl chain; or a combination of these components, or as later defined;
wherein the targeting group is covalently bound to the linker and the linker is covalently bound to the polymer and the targeting group and the linker are as defined herein.

And wherein Compound 10 or 10a is used to manufacture a platinum compound similar to that shown as Compound 9 wherein platinum is bound through two coordination bonds to the amidomalonate chelating group using the processes disclosed herein such that the platinum compound is made from Compound 10 or 10a in a consistent manner at scales ranging from milligrams to scales ≧100 kilograms.

In a further aspect, the present invention also relates to a compound having the chemical structure:

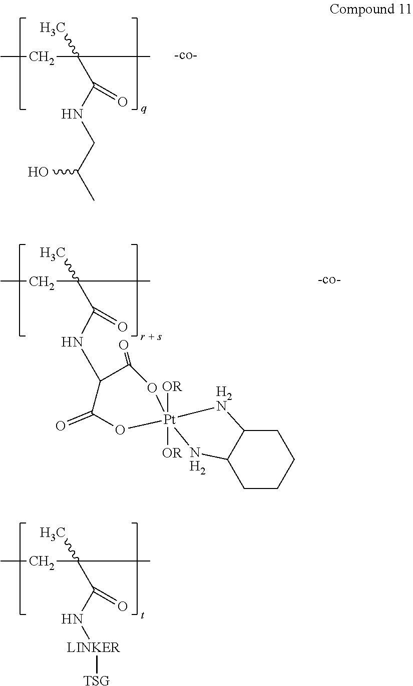

wherein:
‘co’ represents binding of the individual monomer units to form a random copolymer.
q, r, and s represent the molar percentage of the monomer units; such that
q+r+s=100 and
q is ≧50 and q≦95;
r is ≧5 and r≦50;
s is ≧0 and s≦30;
Pt is a +4 oxidation state;
R═H, C₁-C₆ alkyl;
TSG is a tumor seeking group that is capable of targeting cancerous tumor selected from the group of a monoclonal antibody, an antibody fragment, a peptide comprising 2-50 amino acids, a protein, a steroid, a somatostatin analog, a lectin, a folic acid or its derivatives and analogs, vitamin B12, biotin, porphyrin, an essential fatty acid, a bioreductive molecule and a polyanionic polysaccharide; and
Linkers is independently a covalent bond, or an amino acid, or a peptide comprising 2-50 amino acids, or a straight or branched C₁-C₁₀ alkylene chain; or a combination of these components;
or a salt or ester thereof.

An aspect of this invention is a compound as described herein in which all or some Pt atoms are in the +2 oxidation state.

An aspect of this invention is a compound as described herein in which all or some Pt atoms are in the +4 oxidation state.

An aspect of this invention is a compound in which all or some Pt atoms are attached to the two oxygen atoms of the malonate groups.

An aspect of this invention is a compound as described herein in which all or some Pt atoms are attached to oxygen and nitrogen atoms of the peptide spacer group other than the points of attachment shown in the above structure.

A further aspect of this invention are processes for the attachment of platinum atoms to the polymer which provide Compound 11 in a consistent manner and with a high yield with respect to platinum employed.

A further aspect of this invention wherein Compound 11 is purified by a process which separates the polymer from low molecular weight materials.

A further aspect of this invention is in the use of Compound 11 for the treatment of cancer.

A further aspect of this invention is in the use of Compound 11 for the treatment of solid tumor cancers.

A further aspect of this invention is in the use of Compound 11 for the treatment of ovarian cancer.

A further aspect of this invention is in the use of Compound 11 for the treatment of gastric cancer.

A further aspect of this invention is in the use of Compound 11 for the treatment of lung cancer.

A further aspect of this invention is in the use of Compound 11 for the treatment of melanoma.

A further aspect of this invention is in the use of Compound 11 in combination with one or more other anticancer compounds for the treatment of cancer.

A further aspect of this invention is in the use of Compound 11 in combination with one or more other chemotherapeutic for the treatment of cancer.

A further aspect of this invention is in the use of Compound 11 in combination with paclitaxel for the treatment of cancer.

A further aspect of this invention is in the use of Compound 11 in combination with gemcitabine for the treatment of cancer.

A further aspect of this invention is in the use of Compound 11 in combination with 5-fluorouracil and leucovorin for the treatment of cancer

A further aspect of this invention is in the use of Compound 11 in combination with vinorelbine for the treatment of cancer.

A further aspect of this invention is in the use of Compound 11 in combination with paclitaxel for the treatment of cancer wherein Compound 11 and paclitaxel are administered intravenously to the patient once every three weeks.

A further aspect of this invention is in the use of Compound 11 in combination with paclitaxel for the treatment of cancer wherein compound 1 and paclitaxel are administered intravenously to the patient once every two weeks.

A further aspect of this invention is in the use of Compound 11 in combination with paclitaxel for the treatment of cancer wherein Compound 11 is administered intravenously to the patient once every three weeks and paclitaxel is administered intravenously to the patient once every two weeks.

A further aspect of this invention is in the use of Compound 11 in combination with paclitaxel for the treatment of cancer wherein Compound 11 is administered intravenously to the patient once every two weeks and paclitaxel is administered intravenously to the patient once every three weeks.

A further aspect of this invention is in the preparation of a pharmaceutical product comprising, consisting essentially of, or consisting of Compound 11 optionally formulated and intimately mixed with one or more pharmaceutically-acceptable excipients provided as a lyophilized powder in a sterile sealed vial wherein the vial contents are dissolved in a vehicle suitable for administration to a patient just prior to use.

A further aspect of this invention is in the preparation of a pharmaceutical product comprising, consisting essentially of, or consisting of Compound 11 optionally formulated and with one or more pharmaceutically-acceptable excipients dissolved in a vehicle suitable for administration to a patient provided in a sterile sealed vial.

In a further aspect, the present invention also relates to a compound having the chemical structure:

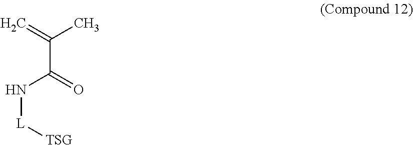

wherein L is a linker group which can be a covalent bond, or an amino acid, or a peptide, or a straight or branched alkyl chain; or a combination of these components, or as later defined;
TSG is a tumor seeking group that is capable of targeting cancerous tumor, non-limiting examples of which are selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharides;
and wherein TSG is either protected by functional groups and methods known in the art or unprotected,
and wherein Compound 12, either protected or unprotected, is caused to react with Compound 3 and either Compound 4 or Compound 7 to form Compound 6 or Compound 10 by the methods described herein or other methods known in the art to form a random copolymer, and the resultant polymer is deprotected as required and reacted with a platinum reagent as described herein to form Compound 5 or Compound 11.

In a further aspect, the present invention also relates to a compound having the chemical structure:

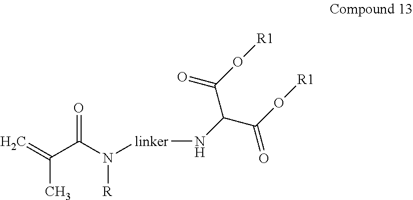

wherein the linker group which can be a covalent bond, or an amino acid, or a peptide, or a straight or branched alkyl chain; or a combination of these components, or as later defined;
and wherein the linker can either be stable in a biological environment or the linker may be subject to chemical or biological breakdown of cleavage in a biological environment;
wherein:
R is H or C₁-C₆ alkyl;
R1 is H or C₁-C₆ alkyl;
and wherein Compound 13 is caused to react with Compound 3 and optionally Compound 12 by polymerization methods described herein or other methods known in the art to form a random copolymer, and the resultant polymer used to form a platinum complex similar in structure to that shown as Compound 1 or Compound 5, wherein said platinum complexes may be used for the treatment of cancer.

In another aspect of this invention, HPMA copolymers are produced which chelate platinum in the +2 oxidation state and square planar geometry and such platinum complexes can be used as anticancer compounds or can subsequently be oxidized to the platinum +4 oxidation state with hexagonal coordination geometry in which the two coordination sites created by oxidation of the metal are filled with monodentate ligands including, but not limited to —OH, Cl, H₂O, (1C-20C)alkylC(O)—, bromoacetate, chloroacetate, chlorodifluoroacetate, dichloroacetate, dichlorofluoroacetate, fluoroacetate, iodoacetate, trifluoroacetate, succinate, benzoate, phthalate, diglycolate, glutarate, 3-methylglutarate, 3,3-dimethylglutarate.

In another aspect, the present invention provides isolated polymers and polymeric platinum complexes. In one embodiment, the isolated polymeric platinum complexes contain at least 80%, 85%, 90%, 95%, 98%, or 99% of an N,O-amidomalonate platinum complex or an O,O′-amidomalonate platinum complex.

Further provided by this invention is a kit comprising a compound or composition as described herein and instructions for formulation and/or use. The use may be therapeutic as described below for use in screening for new chemical entities that have the same or similar activities. The new agent is screened using the in vitro test described below. At the same time, a separate culture is used and the same concentration or dose is added. If the new compound or composition provides the similar activity, it is a potential therapeutic. In addition, new combination therapies can likewise be screened. As is apparent to those of skill in the art, appropriate positive and negative controls such as a cell culture having no additional compound or composition added can be run at the same time.

In a further aspect, this invention further provides, a kit as described above comprising an anticancer compound or composition for use in combination with the compound or composition.

Yet further provided is the use of a compound or composition as described herein for the treatment of cancer.

### BRIEF DESCRIPTION OF THE DRAWING

FIG. 1 depicts the in vivo antitumor effect of a polymeric platinum complex of the present invention alone and in combination with vitamin B12 in tumor bearing mice.

FIG. 2 shows the relationship between reaction conditions and the outcome in preparing poly(HPMA)-GGG-Ame Polymer.

### DETAILED DESCRIPTION OF THE INVENTION

### Definitions

All numerical designations, e.g., pH, temperature, time, concentration, and molecular weight, including ranges, are approximations which are varied (+) or (−) by increments of 0.1, 1.0, or 10.0 as appropriate. It is to be understood, although not always explicitly stated that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a”, “an”, and “the” includes plural references unless the context clearly dictates otherwise.

As used herein, the term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, “alkyl” refers to a saturated (containing no multiple carbon-carbon bonds) aliphatic (containing no delocalized π-electron system), hydrocarbon containing, if otherwise unsubstituted, only carbon and hydrogen atoms. The designation (n₁C-n₂C)alkyl, wherein n₁ and n₂ are integers from one to 6, refers to straight or branched chain alkyl groups comprising from n₁ to and including n₂ carbon atoms. An alkyl group herein may be optionally substituted with one or more entities selected from the group consisting of halo, hydroxy, alkoxy, aryloxy, carbonyl, nitro, cyano, carboxyl and alkoxycarbonyl.

As used herein, “(linker)” abbreviated as “L” or “Lx” refers to an entity which can form covalent bonds to other entities within a molecule such that the linker joins the other entities and provides a spacer or desired separation between the joined entities. The linker can be either be stable in a biological environment or can degrade in a defined manner (for example, because of a pH change, change of oxidative state, or the effect of enzymes) resulting in the separation of the joined entities. Linkers may include, but are not limited to peptides or alkyl groups. Non-limiting examples include one atom, or a group of atoms or molecules such as a single amino acid, oligopeptide or polypeptide, that is used to couple a polyermic backbone to a platinum complex while spatially separating the two entities. Thus, a linker of this invention has an essentially longitudinal axis, that is, it is essentially linear rather than highly branched or clumped, although the structure will, of course, not be exactly linear due to the angular constraints placed on the structure by required bond angles between covalently bonded atoms. It is within the concept of this invention that a linker has no components; in other words, there is no atom of functionality separating the covalent linkage of the polymer backbone with the platinum complex. In one particular aspect, the linker is a single amino acid or a oligopeptide or a polypeptide of varying length, e.g. between 1 and 50, or alternatively between 1, 2, 5 or 10 and 50, or alternatively between 1, 2, 5 or 10 and 40, or alternatively between 1, 2, 5 or 10 and 35, or alternatively between 1, 2, 5 or 10 and 30, or alternatively between 1, 2, 5 or 10 and 25, or alternatively between 1, 2, 5 or 10 and 20, or alternatively between 1, 2, 5 or 10 and 15. Non-limiting examples of peptide linkers are provided in Table 1. The amino acids in the linker may be the same or different and in one aspect, at least one of the amino acids is a glycine. In a specific aspect, the linker is a tri-glycine oligopeptide.

In the structural features described herein as [amino acids]ₐ or [amino acids]ₙ, [amino acids] refers to a (linker) entity comprised of amino acids. The “a” or “n” refers to the actual number of amino acids, i.e., 1, 2, 4 . . . etc., that comprise the [amino acids] linker. An amino acid is a compound that has in its chemical composition, a free amine, i.e. —NH₂, group and elsewhere in its structure a carboxyl, —COOH, group (depending on the milieu the amino acid finds itself in, the amine may exist as —NH₃⁺ and the carboxyl as —COO⁻ that is, the compound is a zwitterion). The use of the plural in (amino acids) is intended to convey the fact that, when “a” or “n” is 2 or more, that is the [amino acids] linker is comprised of two or more amino acids, each amino acid may be the same as, or different than, each other amino acid. For, example, without limitation, if a or n is 4, then [amino acids] consists of 4 individual amino acids, which may be the same or different. The amino acids are bonded to one another through peptide linkages; that is, recurring amide bonds:

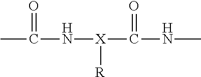

wherein X is a group such as, without limitation, (1C-20C)alkyl (e.g., if X is CH₂, then the amino acid is an α-amino acid), cycloalkyl, aryl, heteroaryl or heteroalicyclic. R can be any group known to those skilled in the art to be compatible with starting amino acids.

It is further understood that, for each iteration of the above peptide bond, X and R may be the same as or different than any other X or R in the chain; i.e., as noted above, the amino acids may be the same or different. The individual amino acids may be natural or synthetic. The natural amino acids include alanine (Ala, A), arginine (Arg, R), asparagines (Asn, N), aspartic acid (Asp, D), cysteine (Cys, C), glutamic acid (Glu, E), glutamine (Gln, Q), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (The, T), tryptophan (Trp, W), tyrosine (Try, Y) and valine (Val, V). A poly(amino acids) group comprised of entirely natural amino acids is also know as an oligopeptide (for shorter chain length) or polypeptide (longer chains). While the truly “naturally-occurring” amino acids have “L” absolute stereochemistry the “L” form, the “D” form and the racemate (50:50 mixture of the two forms) will be considered “natural” amino acids for the purposes of this invention. Any of these may be used alone, in combination with other natural amino acids or in combination with synthetic amino acids, to form the -(amino acids)ₙ-group. Synthetic amino acids useful in this aspect of this invention include any compound with a basic —NH₂ group within 1-20 carbon atoms of a —C(O)OH group.

As used herein, a chelating group refers to a chemical structure which is able to form two or more coordinate bonds to a metal atom with a valency>0. The strength of the coordinate bonds can either provide a stable linkage, whereby the metal can be dislodged only with great difficulty and extreme conditions, or labile, whereby the metal can be displaced from the chelating group under relatively mild conditions, such as those found in the human body.

The preferred polymers of this invention are known as random polymethacrylate copolymers. A “random” copolymer refers to a polymer in which two or more monomers obey Bernoullian distribution in their arrangement in the completed polymer; that is, the sequence of monomers within the polymer chain is random and not predefined. By comparison, a “block” copolymer refers to a polymer in which two or more monomers are present in homogeneous sequences in the completed polymer; i.e., AAA-BBB-CCC-DDD, etc.

A polymer backbone of this invention has an average molecular weight distribution, as determined by the method(s) set forth by methods known in the art of from 1 to 500 kDa, preferably from 5 to 250 kDa and, more preferably, from 5 to 60 kDa.

As used herein, “halo” or “halogen” refers to fluorine (F), chlorine (Cl), bromine (Br) and iodine (I).

As used herein, a platinum cytotoxic agent is a platinum atom in the +2 oxidation state and tetradentate square-planar coordination geometry containing two nitrogen atoms in the cis orientation and two coordination sites for binding to the polymer carriers herein or a platinum atom in the +4 oxidation state and hexadentate octahedral coordination geometry containing two nitrogen atoms in the cis orientation, two axial monodentate ligands and two coordination sites for binding to the polymer carriers herein, and wherein preferred nitrogen compounds bound to the platinum atom are ammonia and diaminocylohexane, or another platinum chelate known in the art as possessing cytotoxic properties in any platinum oxidation state.

As used herein, a primary, secondary or tertiary alkyl amine refers to an RNH₂, an RR″NH or an RR′R″N group, wherein R, R′ and R″ independently represent, without limitation, alkyl, cycloalkyl, aryl, heteroaryl and heteroalicyclic moieties.

As used herein, “cycloalkyl” refers to an all-carbon cyclic or fused multicyclic ring, which, although it may contain one or more double bonds, maintains an essentially aliphatic character; that is, any double bonds present do not interact to form a delocalized π-electron system in the ring. For purposes of this invention, the ring may contain up to 8 carbon atoms. The designation (n₁C-n₂C)cycloalkyl refers to n₁ up to and including n₂ carbon atoms in the ring. As used herein, “fused” means that two cycloalkyl groups share at least one ring atom between them. Compounds such as spiro[4.4]nonane is considered “fused” for the purposes of this invention. More commonly, fused rings share two adjacent carbon atoms. An example of such a fused system is decalin. A “cycloalkylamine” refers to a cycloalkyl group substituted directly on the ring with an —NH₂ group. A cycloalkyl group may optionally be substituted with one or more groups selected from the group consisting of halo, hydroxy, alkoxy, aryloxy, carbonyl, nitro, cyano, carboxyl and alkoxycarbonyl.

As used herein a “tumor-seeking group or ‘TSG’” refers to an entity that is know to preferentially seek out and bond to surface structures on neoplastic cells that do not occur or are expressed to a substantially lesser degree by normal cells or entitles such that the “tumor-seeking” group preferentially accumulates in tumors over normal tissue. Examples of tumor-seeking entities include, without limitation, monoclonal antibodies and/or antibody fragments (including but not limited to anti-CD₂O, anti-CD44, anti-VEGF), proteins and peptides (including but not limited to RGD peptides, Integrin binding peptides), polynucleotides (including but not limited to an RNA aptamer that binds to p68), B vitamins (including but not limited to vitamin B1 (thiamine), vitamin B2 (riboflavin), vitamin B3 (niacin), vitamin B6 (pyridoxine), vitamin B7 (biotin), vitamin B9 (folic acid) its derivatives and analogs, and vitamin B12), steroids, somatosin analogs, lectins, porphyrin, essential fatty acids, bioreductive molecules (including but not limited to nitroimidazoles and their derivatives and N-oxides) and polyanionic polysaccharides (including but not limited to heparin and heparin fragments, hyaluronic acid, carboxymethyldextran, dermatan sulfate).

As used herein, “water-solubilizing” refers to a group that either improves the water solubility of a polymer herein or that confers water solubility on an otherwise insoluble polymer. Water-solubilizing groups useful with this invention include, without limitation, 2-hydroxyethyl, 2-hydroxypropyl, 2,3-dihydroxypropyl, 3-hydroxypropyl, poly(ethylene glycol) and (1C-6C)alkyl groups terminating in SO₃, sulfonato, quaternary ammonium or carboxy.

When designating the composition of a copolymer herein, a percentage (%) refers to the actual number of a particular monomer in the average polymeric molecule of that copolymer. For example, in a co-polymer of formula (Aₚ/B_{q}/Cᵣ) comprising 100 total monomers (in the average polymer chain) where “p” is defined as being from 0 to 25%, “q” as being from 5 to 50% and “r” as being from 50 to 95%, what is meant is that, of the 100 monomers in the average polymer strand, up to 25 are monomer A, up to 50 are monomer B and up to 95 are monomer C. Of course, in all cases, p+q+r=100%. The phrase “m is from 0 to 90% of r” means that a Pt complex modified by “m” in the chemical structure is present in from 1 to 90% of the monomer modified by r. For instance, if “r” is 70% and “m” is 90%, then a Pt complex is appended to 90% of the monomer fragment comprising 70% of the average polymer chain or 63% (90×70) of the total monomers comprising the polymer.

The following formulaic approach is used herein to describe the polymeric compounds of this invention: (polymethacrylate backbone)-(linker)-(leaving chelating group)=Pt=(stable chelating group), wherein the “=” indicates two single covalent or coordinate covalent bonds between the Pt and the attached group. An example, without limitation, is the compound p(HMPA)-GGG-Ama=Pt=DACH. p(HMPA) is the backbone polymer, poly(N-(2-hydroxypropyl)methylacrylamide-co-methacrylamide), GGG is the linker gly-gyl-gyl-, and Ama is the chelating group, amidomalonate, which is chelated to Pt by two coordinate covalent bonds. The Pt is also coordinated by two single bonds to a stable ligand, DACH (1R,2R-diaminocyclohexane). The Pt may also be coordinated to the polymer through other suitable functional groups, particularly nitrogen and oxygen atoms within the linker (as defined). The Pt is also chelated by two single coordinate bonds to the stable ligand, which, again, is DACH. In the above examples, it is understood that (linker)-(leaving chelating group)=Pt=(stable chelating group) groups are appended to methacrylamide monomers only.

Since the exact desired percentage of each of the components in a copolymer is not usually synthetically achievable despite the care taken to use the exact ratio of monomers, the use of the term “approximately” is used to signify that the number shown in the structural formula represents a theoretical value which may vary from that observed by analytical means by as much as ±10%. Thus, in the phrase, “q is approximately 90%,” q in fact can be anywhere from 81-99% (90±10%). The novel processes described herein advantageously provide polymer products which allow for more narrow definition of these ranges in final products, providing products with superior pharmaceutical compliance.

As used herein, the term “cancer” refers to various types of malignant neoplasms, most of which can invade surrounding tissues, and may metastasize to different sites, as defined by Stedman's Medical Dictionary, 25th edition (Hensyl ed. 1990). Examples, without limitation, of cancers which may be treated using the compounds of the present invention include, but are not limited to, brain, ovarian, bowel, gastric, prostate, kidney, bladder, breast, lung, oral and skin cancers, and include carcinomas, sarcomas, s Malignant tumors derived from connective tissue, or mesenchymal cells, malignancies derived from hematopoietic (blood-forming) cells, germ cell tumors, and blastomas.

As used herein, the terms “treat”, “treating” and “treatment” refer to a method of alleviating or abrogating a solid tumor cancer and/or its attendant symptoms. In particular, the terms simply mean that the life expectancy of an individual affected with a cancer will be increased and/or that one or more of the symptoms of the disease will be reduced.

As used herein, “administer,” “administering” or “administration” refers to the delivery of a compound or compounds of this invention or of a pharmaceutical composition containing a compound or compounds of this invention to a patient in a manner suitable for the treatment of a particular cancer.

A “patient” refers to any higher organism that is susceptible to solid tumor cancers. Examples of such higher organisms include, without limitation, mice, rats, rabbits, dogs, cats, horses, cows, pigs, sheep, fish and reptiles. Preferably, “patient” refers to a human being.

As used herein, a “chemotherapeutic” refers to a compound that is useful for treating a disease or disorder in a patient. In particular, a chemotherapeutic, as used herein, refers to a compound that is useful for treating a cancer, especially a solid tumor cancer, in a patient.

As used herein, the term “therapeutically effective amount” refers to that amount of one compound or more than one compound of this invention which when administered to a patient has the effect of (1) reducing the size of the tumor; (2) inhibiting (that is, slowing to some extent, preferably stopping) tumor metastasis; (3) inhibiting to some extent (that is slowing to some extent, preferably stopping) tumor growth; (4) relieving to some extent (or preferably eliminating) one or more symptoms associated with the cancer; and/or (5) extending survival time of the patient.

As used herein, the term “combination” in reference to treatment of a patient refers to the use of a combination of compounds of this invention or one compound or more than one compound of this invention in combination with one or more other anticancer compound to provide a therapeutically effective amount of said mixture of compounds.

As used herein, the term “neoadjuvant chemotherapy” refers to the administration of therapeutic agents prior to the main treatment.

As used herein, the term “adjuvant chemotherapy” refers to additional treatment usually given after surgery where all detectable disease has been removed.

As used herein, the term “palliative chemotherapy” refers to treatment used to extend life and alleviate symptoms.

As used herein, a “pharmaceutical composition” refers to a mixture of one or more of the compounds of this invention with other chemical components such as pharmaceutically acceptable excipients. The purpose of a pharmacological composition is to facilitate administration of a compound of this invention to a patient.

As used herein, a “pharmaceutically acceptable excipient” refers to an excipient that does not cause significant irritation to an organism and does not abrogate the biological activity and properties of the administered composition.

Examples, without limitation, of excipients include calcium carbonate, calcium phosphate, various sugars and types of starch, cellulose derivatives, gelatin, vegetable oils and polyethylene glycols.

As used herein, a pharmaceutically acceptable salt is a salt comprising anions and cations that are suitable for pharmaceutical use. Such salts include, without limitation, chloride, bromide, sulfate, nitrate, various carboxylates as anions, and alkali metal salts, alkaline earth letal salts, ammonium, and mono-di-, and tri-alkyl ammonium salts, and are well known one of skill in the art.

### Platinum Complexes

Platinum(II), also designated as Pt(II), forms four-coordinate square planar complexes with ligands with free electron pairs. In classic antitumor platinum complexes, two of the four ligands are selected so as to be labile under physiological conditions while the other two are stable. The stable groups are most often ammonia or amines at least one of which has an N—H bond capable of hydrogen bonding to the phosphate backbone of DNA. Generally speaking, the am(m)ine groups relate to tumor specificity and drug efficacy while the labile groups relate to stability and toxicity. The novel platinum compounds of this invention are designed to take advantage of various chemical, physical and biochemical features to achieve an optimal relationship between specificity, efficacy and toxicity and to thereby maximize the therapeutic index of the compounds.

The two labile ligands attached to platinum may be, amongst others, a halide, oxygen atom, or a nitrogen atom other than ammonia or amines, such as imines, amides, and heteroaromic nitrogen atoms. In the case of oxygen atoms, hydroxyl, carbonyl, carboxyl and the oxygen in water are known in the art to bind through labile coordinate bonds to platinum.

The remaining two positions on the 4-coordinate Pt(II) atoms of platinum anticancer agents are ammonia or amines. A broad range of stable nitrogen-containing ligands is within the scope of this invention. Presently preferred stable ligands are either ammonia or diamines of which the following are representative, non-limiting examples:

NH₃, CH₃CH₂NH₂, CH₃CH₂CH₂NH₂, CH₃CH(NH₂)CH₃, CH₃CH₂CH(NH₂)CH₃, H₂NCH₂CH₂CH₂NH₂, H₂NCH₂CH₂CH₂CH₂NH₂, H₂NCH₂CH₂CH(CH₃)CH₂NH₂, (HOCH₂)₂C(CH₂NH₂)₂,

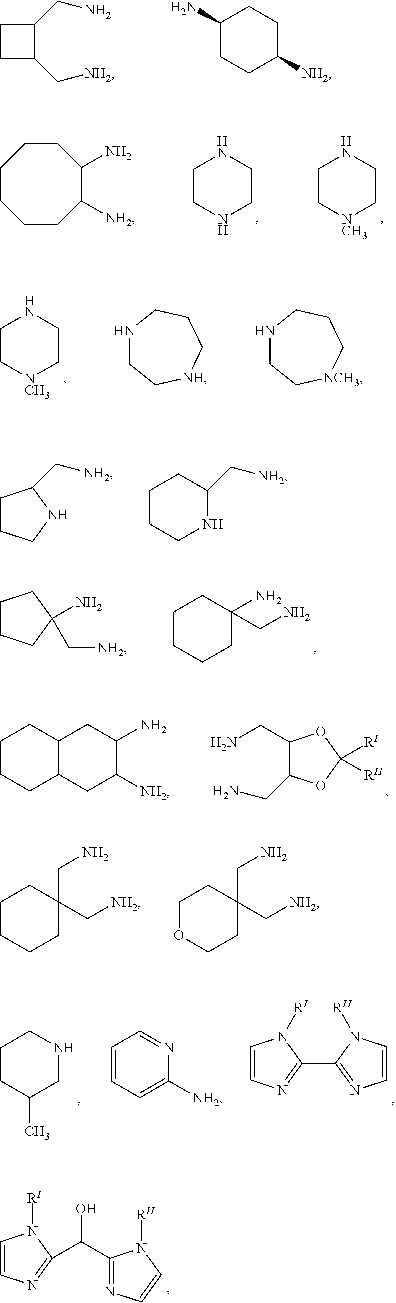

and NH₂CH(R^{III})CH(R^{IV})NH₂, wherein R^{I} and R^{II} are independently (1C-6C)alkyl and R^{III} end R^{IV} are independently hydrogen, (C1-C6)alkyl,

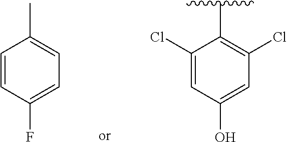

Monomeric platinum complexes include as ligands one or amines or diamines as disclosed above and/or chloride, nitrate, sulfate, water, and such other ligands, and are well known to one of skill in the art.

The preferred diamine of this invention is 1,2-diaminocyclohexane (DACH). Many of the examples given, including DACH contain one or more chiral center, i.e. they possess two or more stereochemical configurations. In the case of DACH, three stereoisomers are known, which are often designated by the Cahn-Ingold-Prelog (CIP or R—S) nomenclature system as R,R′-, S,S′- and R,S-stereochemical forms. The R,S-form is not suitable for the formation of platinum chelates and DACH platinum pharmaceuticals generally utilize either the R,R′- or S,S′-stereoisomers, and most usually the R,R′-isomer, or mixtures of R,R′- and S,S′-. Given pharmaceutical principles well known in the art, it is to be expected that the R,R′- or S,S′-DACH platinum stereoisomers give rise to differences in efficacy and toxicity when administered to patients. Similarly, in the other diamines listed above, where the diamine possesses one or more chiral centers, differences in pharmacological properties are to be expected from individual stereoisomers. Therefore it is desirable that chiral platinum pharmaceutical active ingredients are either single stereoisomers or well-controlled mixtures of isomers.

HPMA polymer-platinum compounds similar to those described herein have previously been described by ourselves and colleagues, most notably in U.S. Pat. No. 5,965,118; U.S. Pat. No. 6,692,734; U.S. Pat. No. 7,166,733; and US2005038109. While these patents acknowledge stereoisomeric forms of the platinum species, the necessity for high stereoisomeric purity of the platinum chelate in the product is not fully appreciated, especially given the possibility that the manufacturing process could give rise to racemization. It is therefore important that the platinum reagent used to make the HPMA polymer-platinum compound has a well-defined and well-controlled stereochemical purity, and that the processes, as outlined in this application, minimize any racemization of the stereoisomers so that the stereochemical profile and purity of the product is controlled by the stereochemical profile and purity of the platinum reagent.

The process for the addition of platinum to the polymer involves the creation of a platinum reagent containing the stable bidentate amine chelate (on two of the four Pt(II) coordination positions) and a highly labile pair of ligands in the remaining two coordination positions, then reacting this reagent with the polymer after removing all protecting groups from the coordination sites on the latter. A series of ligand exchange steps then ensues resulting in N,O-binding of the platinum to the chelator and to the peptide linker, as outlined in U.S. Pat. No. 6,692,734 and U.S. Pat. No. 7,166,733.

For oxygen and nitrogen atoms forming labile coordinate bonds with platinum, the two atoms may be chemically linked by a suitable short spacer such that, when both atoms are bound to platinum, a chelate link is formed. Molecules which form labile chelates of platinum(II) include oxalate, C-substituted malonate, and peptides. For biological activity, particularly anticancer activity (i.e. platinum binding to the DNA of tumor cells) coordinate bond lability is an important requirement as the platinum has to be released into an active form in order to react with and bind to DNA.

The 6-member ring chelates are expected to generally be more labile than the corresponding 5-member ring complexes. The chemical characteristics of 6-member rings compounds, however, will be susceptible to the same structural feature manipulations as the 5-member ring compounds. That is, 6-member ring imides are expected to be more labile than the corresponding amides and the corresponding alkyl sulfonamides, aryl sulfonamides, phosphamides, etc. are also expected to provide compounds with a useful balance of stability and lability. While the 6-member chelates may be formed from any combination of nitrogen, oxygen and sulfur (except as noted above), the 6-member N,O complex is also presently preferred. In fact, it is expected that, with amides having at least one hydrogen on the amide nitrogen and an oxygen in position to form a 5- or 6-member ring, an O,O complex may initially form but the amide will deprotonate to give a softer and therefore preferable ligand for Pt such that the O,O chelate rearranges to the N,O chelate. In the case of the 5-member ring O,O chelate, it is expected that, even in the presence of a deprotonated amide, an equilibrium may exist between the 5-member O,O and the 6-member N,O complexes.

Some specific examples of 6-member ring Pt complexes follow. As with the 5-member ring examples, the examples below are for illustrative purposes only and are not intended, nor are they to be construed, to limit the scope of this invention in any manner.

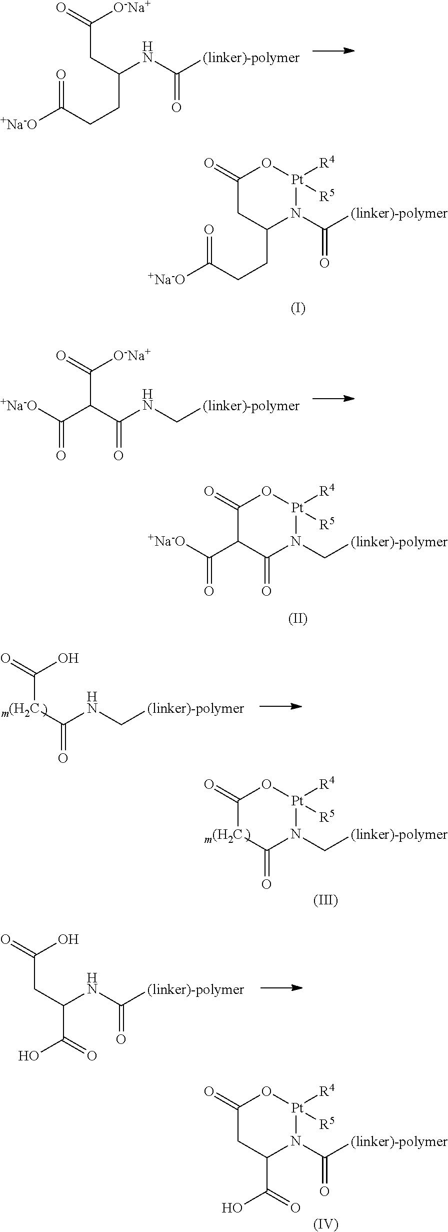

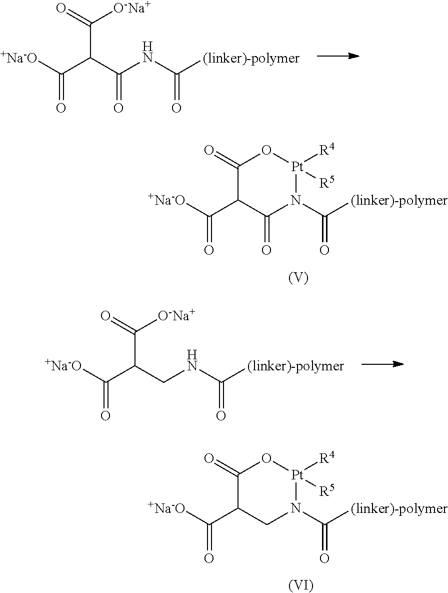

R⁴ and R⁵ are both NH₃ or, together (i.e., R⁴—R⁵) are 1R,2R-diaminocyclohexane, or are 1S,2S-diaminocyclohexane, or are a controlled mixture of 1R,2R-diaminocyclohexane and 1S,2S-diaminocyclohexane.

Compound (I) is an N,O-chelate of a carboxyl group and the nitrogen of n-glutamate, (II) is an N,O-chelate of a carboxyl and an amide with a second carboxylate group appended to the ring, (III) is an N,O-chelate of a carboxyl and an amide without an appended carboxylate; (IV) is and N,O-chelate of a carboxyl and the amide nitrogen of aspartate; (XVI) is an N,O-chelate of an carboxyl and an imide nitrogen and (V) is a chelate of a carboxyl and the amide nitrogen of 2-amidomethylmalonate.

The preferred compounds of the current invention utilize an HPMA (hydroxypropylmethacrylamide) copolymer backbone. HPMA is copolymerized with one or two other methyacrylamide monomers to yield a random copolymer in which the majority of the monomer units are HPMA and a minority of the monomer units contain other functionalities. In order to bind platinum to the polymer, a chelating group, either in a protected or unprotected form, has to be present on the monomer prior to polymerization such that the chelating group can be utilized to bind the metal after polymerization. The preferred compounds of this invention have a peptide linker, most preferably triglycine, connecting the carboxylic acid of the methacrylate polymer to the chelating group, most preferably aminomalonic acid. Platinum binding to this chelating group may take place through the two oxygen atoms of a malonate chelating group, one malonate oxygen atom and adjacent amide nitrogen atom, or through binding to an oxygen atom and a nitrogen atom in the peptide chain. Binding of two or more platinum atoms to a single peptide/chelating group may occur through a combination of binding to the peptide and O,O′- of the amidomalonate or N,O of the amidomalonate. N,O chelates are presently preferred. Additionally, the platinum(II) polymer complexes of this invention may be used for the treatment of cancer or they may be used for the preparation of platinum(IV) complexes by oxidation of platinum(II) through processes and reaction conditions known in the art. Platinum(IV) complexes are also known to be anticancer agents. It is believed that platinum(IV) complexes are stable and do not possess anticancer activity per se, but can be reduced to platinum(II) in vivo and exert an anticancer effect once reduced. The platinum(IV) compounds of this invention are considered to be effective as anticancer agents, and this invention is not restricted to any one possible mechanism of action.

The various structural modifications of platinum complexes disclosed herein can also be manipulated so as to take advantage of differences in the physiological/biochemical environment in tumors compared to normal tissue. For example, the pH of normal tissue and of circulating blood is 7.4. The pH within tumors is often an order of magnitude lower, i.e., 6.2-6.5. The lower pH results from a lower oxygen partial pressure within tumor cells. The cells adapt to this by switching to anaerobic metabolic pathways which results in an increase in lactate production and concomitant reduction in extracellular pH. A platinum complex involving weakly basic ligands such as imines should provide chelates that are relatively stable at essentially neutral pH, i.e., 7.4, but are substantially less so at one pH unit lower where the more acidic medium can protonate the ligand resulting in the cleavage of the coordinate bond.

It is known in the art that macromolecules offer some benefit towards targeting of anticancer compounds to tumors by virtue of the enhanced permeability and retention (EPR) effect (Maeda H., Tumor-Selective Delivery of Macromolecular Drugs via the EPR Effect: Background and Future Prospects, Bioconjug Chem. (2010) 21(5):797-802.

Furthermore, the above molecule may comprise an active tumor-seeking group (as opposed to the passive EPR-related accumulation also expected from the polymeric compounds of this invention) in yet another presently preferred embodiment of this invention.

The stability of a polymer-(linker)-Pt-complex of this invention and, thus, its ability to remain intact until it reaches a target tumor is dependent on several factors: the distance between the polymer backbone and the Pt complex(es), which correlates with the length of the linker, the composition of the “stable” ligand(s) and the composition of the leaving-ligand(s).

Table 1 shows the effect of the distance from the backbone polymer to the Pt complex and of the structure of a linker, e.g., the stable ligand/chelate on the stability of the molecule as represented by the percent small molecule Pt species, i.e., a Pt species no longer tethered to a polymeric backbone, released at 3 and 24 hours. The compound tested comprised a poly(N-(2-hydroxypropyl)methacrylamide (10%)-co-methacrylamide (90%)) backbone polymer and either cis-diammine or 1R,2R-diaminocyclohexane (DACH) as the stable ligands/chelate of the Pt complex. The leaving-ligand comprised an N,O bidentate ligand with amidoaspartate (Asp) or amidomalonate (Ama). As can be seen, Ama complexes are more stable than Asp complexes, the further from the backbone the complex is, the more stable it is and, cis-diammine stable-ligand complexes are more stable than DACH chelate complexes.

TABLE 1

| | Percent small platinum | | Percent small platinum | |
|---|---|---|---|---|
| | released from cis-diammine | | released from 1R,2R-DACH | |
| Linker between | Pt(II) complex at 37° C. in PBS | | Pt(II) complex at 37° C. in PBS | |
| polymer and complex | 3 hours | 24 hours | 3 hours | 24 hours |
| No linker-Ama=Pt= | 4.1% | 23.8% | 17.6% | 66.3% |
| Gyl-Gly-Ama-Pt= | 0.6% | 3.2% | 1.5% | 7.6% |
| Gly-Gly-Asp=Pt= | 3.9% | 11.0% | 4.4% | 12.6% |
| Gly-Phe-Leu-Gly- | 0.7% | 2.1% | 0.7% | 2.4% |
| Ama=Pt= | | | | |
| Gly-Phe-Leu-Gly- | 1.5% | 4.7% | 2.0% | 6.5% |
| Asp=Pt= | | | | |

The difference in percent Pt release due solely to the change from NH₃ to DACH as the stable ligand suggests that a substantial level of control over delivery of small molecule species to a target tumor is possible based on this structural variation alone.

While (amino acids) linkers are presently preferred, other linkers are also within the contemplation and scope of this invention. For example, without limitation, a poly(ethylene glycol) with up to 10 ethylene units could be coupled with L-alanine or L-alanine-L-valine as the chelating agent. Such linkers would also be water-solubilizing. In the latter case, L-alanine-L-valine has been shown to be sensitive to cleavage by the enzyme thermolysin and thermolysin-like enzymes have been reported as being expressed by a number of tumor types (Suzawa, et al., J. Control Release, 2000, 69(1):27-41). Polymers containing such linkers can be monodisperse (all strands essentially the same size) or polydisperse (range of strand sizes) although particles with low dispersities, which are more easily pharmaceutically characterizable, are presently preferred.

The linkers of this invention may, but need not necessarily, be biodegradable. While biodegradability may be a desired characteristic in some cases, it has been found to not be necessary for the release of active Pt species. While not being bound to any particular theory, it is postulated that the bound Pt complex, despite the steric constraints at the N,O-complexing site, is still susceptible to hydrolytic cleavage at the linker thus releasing the diaqua small molecule Pt complex.

Other linkers useful for the preparation of molecules of this invention include co-aminoalkanoic acids of from 1 to 20 carbon atoms wherein the terminal amino group is reacted with a pendent carboxylic acid group of a backbone polymer and the carboxylic acid group at the other end of the linker is used to form the chelate with Pt. The carbon atoms between the amino and the carboxyl group can be further substituted with water-solubilizing groups such as, without limitation, hydroxyl and/or guanidino.

The cytostatic or cytotoxic Pt(II) complexes owe their pharmacological activity to their ability to form adducts with DNA. Pt(IV) complexes do not form such adducts and are relatively biologically inert. Their very inertness, however, can be used to advantage. That is, Pt(IV) complexes should be amenable to oral administration and should be capable of entering and passing through the circulatory system unaffected. They then could circulate until they passively encounter a target tumors. Then, upon entering the tumor, they could convert to the active Pt(II) species. The stability of Pt(IV) complexes is also expected to permit formulation in aqueous compositions rather than, say, requiring a lyophilized powder that must be reconstituted.

The environment within many tumors is well-suited to effect the conversion of Pt(IV) species to Pt(II) species. That is, in vivo reduction of Pt(IV) to Pt(II) is believed to occur through a reductase enzyme in the presence of a reducing agent/anti-oxidant such as glutathione or ascorbic acid, a milieu commonly encountered in the extra-cellular environment of tumors.

Pt(IV) complexes can be prepared by oxidation (e.g., hydrogen peroxide or chlorine) of Pt(II) complexes in the presence of ligands such as acetate which will occupy the axial positions when the 4-coordinate Pt(II) species is oxidized to the 6-coordinate Pt(IV) species. It is expected that, using relatively mild oxidation techniques, the Pt(IV) complexes of any Pt(II) complex described herein can be readily obtained.

While drug delivery to tumors is generally improved by using a water-soluble backbone polymer or by appending water-solubilizing groups to a polymer backbone, delivery can be further enhanced by appending specific tumor-targeting groups to the backbone polymer. The rationale is that, by virtue of the affinity of the targeting group for a receptor exclusive to, or at least over-expressed by, neoplastic cells or for some other specific tumor characteristic, the concentration of the polymer in the vicinity of the tumor is increased compared to that in the vicinity of normal cells/tissues that do not exhibit the characteristic. Even if the affinity is low, such as in the case of a low binding constant (either intrinsically low, or diminished because the targeting group is bound to the polymer, and so is less able to interact with the target characteristic), increased concentration and/or selectivity of the drug is still to be expected. In addition, when the characteristic is a receptor and a single polymer strand of a drug hereof contains several targeting groups, there may be several receptor-ligand interactions for each polymer strand, amplifying the affinity of the polymer. This is known as the ‘multi-valency’ effect. Targeting groups such as folate and vitamin B12 are expected to be capable of taking advantage of this phenomenon.

It has been recognized that rapidly dividing cells undergo receptor-mediated uptake of certain vitamins. In particular, many types of neoplastic cells contain receptors which mediate rapid absorption of folic acid (Antony, J. Biol. Chem., 1985, 260(28):14911-7). Thus, linking folic acid to chemotherapeutic agents has been recognized as a useful method for increasing the tumor concentration of chemotherapeutics (Leamon, C. P., Low, P. S., Drug Discov. Today, 2001, 6(1):44-51; Wang, S., Low, P. S., J. Control Release, 1998, 53(1-3):39-48), U.S. Pat. Nos. 5,108,921; 5,416,016; 5,635,382; 5,820,847; 5,688,488). Thus, it is expected that folate, appended to the backbone of a polymer-linker-Pt complex of this invention, will likewise assist in the accumulation of the compounds in target tumors.

Tumor-targeting peptides are also presently of particular interest. Several tumor-targeting peptides have been described for use in radionuclide imaging (Behr, T. M., Gotthardt, M., Barth, A., Behe, M. Q., J. Nucl. Med., 2001, 45(2):189-200), and in targeting of 1:1 peptide-chemotherapeutic agent conjugates (Schally, A. V., Nagy, A., Eur. J. Endocrinol., 1999, 141(1):1-14). Other targeting peptides include, without limitation, somatostatin analogs and vasoactive intestinal peptide (VIP). VIP has shown promise for targeting colorectal cancer (Rao, et al., Nuclear Medicine and Biology, 2001, 28:445-450). These and other tumor-targeting peptides are likewise expected to be amenable to attachment to the polymer backbone of the compounds of this invention and to assist in the targeting of the compounds to tumors.

Essential fatty acids such as docosahexaenoic acid (DHA), while used by virtually all types of cells, are particularly avidly taken up by tumors, probably due to the uncontrolled growth characteristic of neoplastic cells. It is expected that fatty acids appended to the polymeric backbone of a compound of this invention will be useful to further facilitate the targeting of tumors.

Bioreductive molecules such as, without limitation, nitroimidazoles are known to bind to hypoxic tissue but not to normoxic tissue (P. Wardman, Radiat. Phys. Chem., 1987, 30:423; Chapman, J. D., et al., Advanced Topics on Radiosensitizers of Hypoxic Cells, A. Breccia, C. Rimondi, and G. E. Adams, eds., Plenum Press, New York, pp. 91-103). That is, nitroimidazoles are reduced by reductases present in virtually all cells but in normoxic tissue, the reaction is rapidly reversed and the compound can be excreted. In hypoxic tissue, however, the reduced species is converted to an entity that forms covalent bonds with endogenous nucleophiles, thereby trapping the compound in the tissue. While some neoplastic tissues are similar to hypoxic tissues in their ability to trap bioreductive agents, they are different from hypoxic tissues in that the latter do not exhibit the EPR effect and it is expected that large molecules, such as the polymer-bound Pt complexes of this invention, when modified with bioreductive groups will preferentially invade and accumulate in tumor tissues even in the presence of hypoxic tissue. It is expected that a polymer-(linker)-Pt complex/bioreductive agent will be irreversible immobilized in the tumor after which small molecule Pt species can be released over virtually any desired time frame.

It is presently a particularly preferred embodiment of this invention, with regard to any of the compounds of this invention wherein the compound comprises a -(linker)-polymer group, as shown above, or a -(linker)-R¹⁰ group wherein R¹⁰ is a polymer, as seen below, that the linker group contains one or more additional Pt chelates at a location or locations intermediate between a terminal chelate (e.g., the compounds above) and the point of attachment of the linker to the polymer backbone. The additional Pt chelates may have the same or different structures than the terminal chelate and/or than each other.

### Syntheses of HPMA Polymer Platinum Complexes

HPMA polymer platinum complexes and methods of making them have been described in U.S. Pat. Nos. 5,965,118, 6,692,734, 7,166,733 and US Patent Application 20050038109 which are incorporated, including any drawings, by reference as if fully set forth herein.

The synthesis of the presently preferred embodiment of this invention, i.e., polymer-backbone-containing compounds having poly(amino acids) linkers appended thereto, the linker each having a terminal Pt chelate and a proportion of them also having a second Pt chelate located between the terminal chelate and the point of attachment of the linker to the polymeric backbone, is also provided in the Examples section.

The method of synthesis of the HPMA platinum complex exemplified by the structure Compound 1 was described previously in the aforementioned patents and patent application, and in journal publications. It involved the addition of a substantial excess quantity of DACH platinum dinitrate, pretreated to generate the corresponding reactive diaqua platinum complex, with a solution of the polymer (exemplified by Compound 2, which had been pretreated to remove the ester groups protecting the carboxyls). pH adjustments, and addition of salts and Chelex resin was employed to control the reaction. Platinum initially forms an O,O′-chelate with the malonate group, then rearranges on pH adjustment to form more stable N,O-chelates. Process conditions control the relative amounts of N,O- and O,O′-chelates formed. The product was isolated by tangential flow filtration. This process was inefficient, having a low yield with respect to the platinum reagent, gave inconsistent product, and was not amenable to scaleup. An improved process, as exemplified in the examples section, involves using a pH-stat to maintain pH control and the elimination of Chelex resin. The new process provides a higher yield of product with respect to platinum reagent, a product which is more consistent batch-to-batch, and a process which is scalable to multikilogram levels.

For the synthesis of the polymer exemplified by Compound 2, the process described in the aforementioned patents involved the construction of a methacrylate monomer which contains the peptide linker or a portion of the peptide linker terminated by an active ester, typically a p-nitrophenyl ester. Copolymerization with HPMA results in formation of an intermediate polymer which is reacted in an additional step with diethyl aminomalonate or an N-aminoacid or N-peptide derivative of diethyl aminomalonate to complete the peptide linker and attach the chelating group to the polymer.

The preparation of the polymer by this route is exemplified by the following schematics

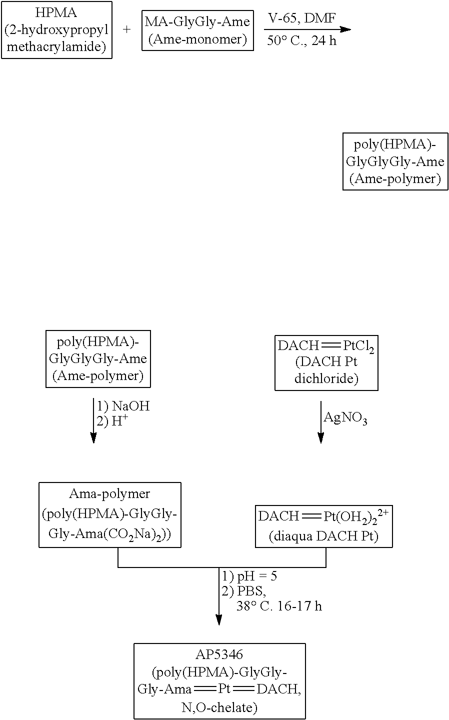

Compound 4 (MA-Gly-Gly-Gly-Ame) is the new and inventive monomer for the cost-effective and scalable process described herein for the manufacture of a polymer platinum therapeutic agent in which the platinum chelating group is an amidomalonate. The following schematics describe methods for making compound 4. Other possible methods will be obvious to those skilled in the art.

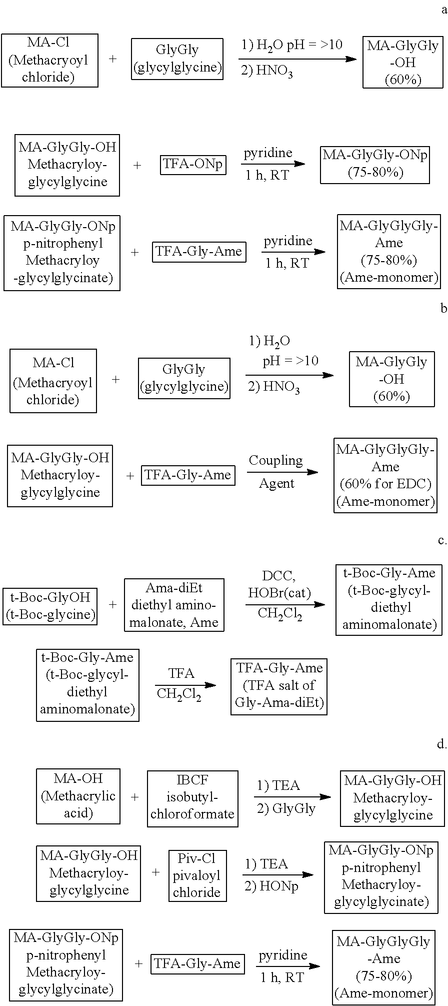

Similarly, the HPMA monomer can be made by various process that are either known in the art or new, as exemplified by the following schematics:

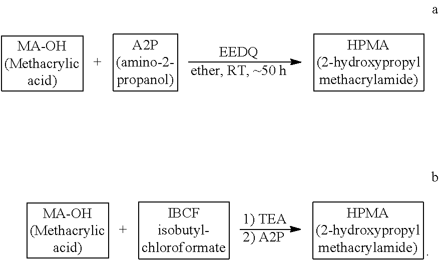

The previously reported process for the preparation of compound 2 involved a process providing low yields, poor scalability, and higher cost of goods, thus making it undesirable for commercial manufacture of the polymer platinum product exemplified by Compound 1. The following schematics outline the previously reported process:

TFA-Gly-Ame

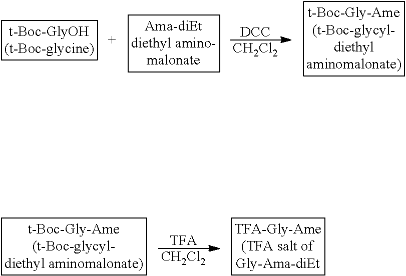

Two Methods for the Preparation of MA-Gly-Gly-ONp

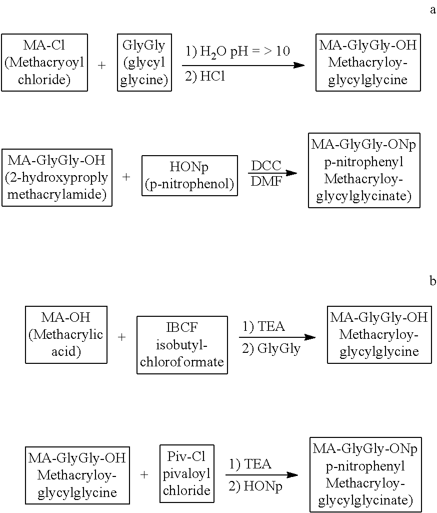

Poly (HPMA)-GlyGly-ONp

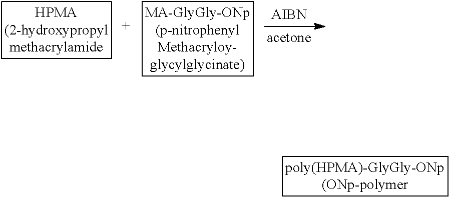

Compound 2 (poly(HPMA)-Gly-Gly-Gly-Ame)

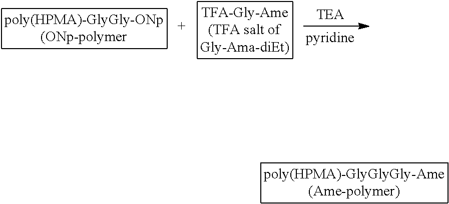

The improved process for the preparation of polymer exemplified by Compound 2 first involves the synthesis of a monomer, exemplified by Compound 3, in which the complete linker and complexing group are attached to a methacrylate unit, and the carboxyls of the complexing group are protected as alkyl esters. Thus polymerization of the novel monomer exemplified by Compound 3 with HPMA monomer in one step. The process can be conducted in a variety of organic solvents and with a variety of different free-radical initiators. For a given solvent and free radical initiator, batch-to-batch control and consistency of polymer molecular weight and polydispersity are provided by process control of temperature, concentration, and rates of addition. As exemplified in the examples section, this process provides the polymer with well controlled characteristics, in good to high yield.

The polymerization of compounds 3 and 4 to give compound 2 can be conducted using polymerization processes well known in the art. Preferred processes are free-radical processes including controlled polymerization processes. A variety of free radical initiators can be used, for example, as detailed in E. T. Denisov, T. G. Denisova, T. S. Pokidova, Handbook of Free Radical Initiators, 2003 John Wiley & Sons, Inc., ISBN: 9780471207535). Preferred are azo compounds including water soluble compounds, exemplified by VA-044, VA-046B, V-50, VA-057, VA-060, VA-061, VA-067, VA-080, VA-086.

Organic soluble compounds, exemplified by V-70, V-65, V-601, V-59, V-40, VF-096, V-30, VAm-110, VAm-111, and macro azo initiators, such as VPE-0201, VPE-0401, VPE-060. Most preferred are the initiators listed in the following table:

| Common | | | |
|---|---|---|---|
| Abbreviation(s) | Chemical Name | Structure | numbers |
| AIBN | 2,2′-Azobis(2- methylpropionitrile) | 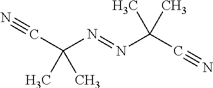 | V-60 VAZO64 |
| ABVN/ACCN/A DVN | 2, 2′-Azobis-(2, 4- Dimethylvaleronitrile) | 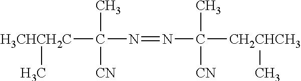 | V-65 VAZO52 |
| ABCN | 1,1′-Azobis(cyclohexane- carbonitrile) | 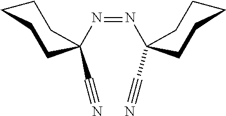 | V-40 VAZO88 |

Compound 2 can also be produced from Compounds 3 and 4 by controlled or living polymerization processes such as Atom Transfer Radical Polymerization (ATRP) or reversible addition-fragmentation chain transfer polymerization (RAFT) (for example, G. Moad, S. H. Thang, RAFT Polymerization: Materials of The Future, Science of Today: Radical Polymerization—The Next Stage, Aust. J. Chem. 2009, 62, 1379-1381).

The preceding overview has focused on the polymerization of compounds 3 and 4 to provide compound 2. The scope of this invention is not limited to processes involving only compounds 3 and 4, but includes other monomers as outlined earlier in this application.

The initial part of the new preparation of Compound 1 from its Ame-polymer precursor is a four-part process with subsequent purification and lyophilization steps. While no intermediates are isolated, two in situ purifications are performed. The platinating solution of the dinitrato salt of DACHPt(OH₂)₂ 2+ is prepared by heating a stirred mixture of DACHPtCl₂ and AgNO₃ with a trace of nitric acid. The nitric acid and heating steps are performed to prevent any μ-hydroxo Pt species forming. Slightly less than 2 equiv of AgNO₃ are used otherwise the filtered solution becomes cloudy with slow forming AgCl.

Saponification of the Ame-polymer at pH 12.6 has been found by ¹H NMR to proceed very quickly (complete within a few minutes). To remove any non-polymer-bound ions that would allow nonproductive electrostatic interactions with the platinating species, the solution can be neutralized using mixed bed ionexchange resin.

The saponified polymer solution can be combined with the platinating solution at pH 7.4. This pH is chosen to give a combined reaction mixture with a pH of approximately 5. This pH control is important because at high pH levels the diaqua platinum species (pKa1) 5.4, (pKa2) 7.2 at 27° C. becomes significantly deprotonated, yielding the less reactive mono- or unreactive dihydroxo species. The pH can be kept above 3 to ensure that no decarboxylation (pH<2) takes place. A pH near 5 is preferred so that a high proportion of the amidomalonate groups are doubly deprotonated (pKa values of approximately 2.8 and 5.6). Thus, at a pH of approximately 5.0-5.4, the predominate Pt species will be the reactive DACHPt(OH₂)₂ 2+, decarboxylation is avoided, and the amidomalonate is at least singly deprotonated with a substantial fraction of the doubly deprotonated species. These factors are all seen to facilitate the desired chelate formation and inhibit undesired or unproductive side reactions. The solution of the saponified polymer is treated with almost 2 equiv of the platinating solution. Several stoichiometries have been tested and this value was chosen as it resulted in materials with the most desirable biological activity. A reaction time of 2 h has been found to be ample for formation of the predominately O,O′-chelate solution. Before proceeding to the O,O′- to N,O-chelate conversion step, the reaction mixture can be treated with a metal chelating ion-exchange resin (Chelex Resin, biotech grade). Since the O,O′- to N,O-chelate conversion is performed in 110 mM NaCl and since remaining DACHPt(OH₂)₂ 2+ converts to the poorly soluble DACHPtCl₂ at this concentration, the Chelex resin was can be to scavenge any remaining free platinum species. This resin does not allow the polymer platinum conjugate to penetrate because it has a nominal molecular weight cutoff (NMWCO) of 1 kDa. Treatment for 90 min is sufficient to remove any such Pt species as confirmed by ICP analysis. The filtered solution of mostly O,O′-chelate (approximately 85% O,O′-, 15% N,O-) is converted in situ to the N,O-species, as described, in PBS at pH 7.4 and 38° C. These salts are chosen for their general biocompatibility. The chloride concentration is close to physiological, and it is expected and desired that any labile platinum species under such concentrations would be lost in the chelate conversion. The phosphate quantity is chosen not only to give well buffered solutions but also to account for the fact that some may also react with any free Pt species. While O,O′- to N,O-chelate conversion is essentially 100% at pH 7.4, it is interesting to note that the O,O′-chelate can be re-formed by treatment of the AP5346 N,O-chelate solution at pH 3 at 38° C. After 3 h under these conditions a mixture of 85% O,O′-, 15% N,O- can be obtained. Furthermore, when the mixture of mainly O,O′- and some N,O-chelate (prior to PBS treatment for conversion to N,O-species) is treated in situ at pH 3 (38° C., 3 h), the platinum converts almost entirely back to the O,O-form. Greater than 98% O,O-chelate can be achieved. Increasing the reaction time may not increase the percentage of O,O′.

Purification of the Compound 1 crude product can be performed by TFF using a polyethersulfone (PES) membrane with a 5 kDa NMWCO. A TFF-based purification was chosen rather than preparative SEC because it is linearly scalable from mg to multikilogram amounts. Concentration of the purified solution to 10% w/v followed by lyophilization produces a cake of the drug substance. Less concentrated solutions produced a material which was difficult to handle due to static.

This process had several disadvantages from a cost perspective as well as scalability. A more cost-effective and scalable process was sought for the platination step. Both mixed bed and Chelex ion exchange resins are expensive and elimination of these resins would be advantageous. An improvement of the reaction yields in terms of weight and/or platinum was important because of the high cost of platinum. Last, a more concentrated process to allow for larger scale-up was considered to be necessary.

The formation of the DACHPt(OH₂)₂ 2+ platinating solution for the improved synthesis can be achieved by heating DACHPt-(NO₃)₂ in low pH water for 1 h. The diaqua species readily forms, and there is no need for overnight heating, filtration, and in-process testing for platinum concentration by ICP as there is in the initial method. Additionally, significantly less platinum can be used (approximately 40% reduction), while obtaining the same quantities of platinum in the final product. The saponified polymer solution can be prepared in the same way in both processes; however, neutralization is performed using 5% HNO₃ in place of the more expensive mixed bed ionexchange resin. Upon combination of these solutions the pH can be held constant at 5.2 for 2 h using an autotitrator loaded with NaOH. In this process, however, the resulting (mainly) O,O′-chelate solution can be converted to the N,O-chelate by merely increasing the pH to 7.4 and heating at 38° C. for 17 h. In the initial process, where a large excess of platinum is used, the same process can be performed by overnight heating in PBS. The high chloride concentration prevents a significant quantity of platinum from binding to the polymer. In contrast, the lack of PBS in the improved chelate conversion process can result in the reduction of the amount of platinum used, such that all or most of the platinum which is added binds to the polymer. Therefore there is no need for the addition of Chelex resin for removal of excess free platinum. Isolation and analysis of the N,O-chelate at this stage of the synthesis shows that by 1H and 195Pt NMR, this N,O-chelate is identical to the final platinated product. An additional TFF purification step is an optional additional step for further purification. This involves a short treatment with PBS (200 mM Cl—, 80 mM PO43-) at 38° C. for 4.5 h, and then subjected to a second TFF.

It is believed that the short PBS treatment causes rearrangement and/or removal of small concentrations of highly labile Pt species to produce the lower Pt releasing species. The resulting solution can be lyophilized to produce compound 1 product which is identical to compound 1 prepared from the original process except that it is white/off white in color. Compound 1 prepared from compound 2 prepared by the original process has a brown color. A large number of batches of compound 1 have been prepared to confirm that product can be prepared consistently and conveniently scaled to larger batch sizes and, and it was found that the analytical results for these batches of compound 1 usually appear within a relatively narrow range inside those specifications.

Both methods of manufacture of compound 1 produce materials which are identical by 1H NMR spectroscopy.

In summary, the process improvements which are the subject of this invention involve the elimination of Chelex resin, instead using precise pH control using a pH stat or similar device, and additional TFF purification to produce a product which has excellent batch-to-batch consistency and which is scalable.

### Biological Evaluation

Biological evaluation can be carried out with the methods as described below. A murine study was conducted which confirmed that Compound 1, as made by the process described herein (in which Compound 2 is made according to the process described in Example 6A and Compound 1 is made according to the process described in Example 8B and 9) has a safety profile identical to that of Compound 1 made by the processes described previously (as described in U.S. Pat. No. 7,166,733).

A clinical monotherapy dose-ranging study was conducted in recurrent ovarian cancer patients using Compound 1 made by the process described previously in U.S. Pat. No. 7,166,733. A total of 26 patients were enrolled into the study, which yielded significant safety and efficacy data for Compound 1. The study was then extended to included 9 patients that received Compound 1 made by the improved and novel process described herein. For patients treated at similar dose levels of Compound 1, efficacy (as demonstrated by stabilization of tumor size) and safety profile were also similar in both groups.

### Pharmaceutical Applications, Kits and Preparations

A compound of the present invention can be administered as such to a human patient or can be administered in pharmaceutical compositions in which the foregoing materials are mixed with suitable excipient(s). Techniques for formulation and administration of drugs may be found in Remington's Pharmacological Sciences, Mack Publishing Co., Easton, Pa., latest edition.

### Routes of Administration

Suitable routes of administration may include, without limitation, oral, rectal, transmucosal, intestinal administration, intramuscular, subcutaneous, intramedullary, intrathecal, direct intraventricular, intravenous, intravitreal, intraperitoneal, intranasal, or intraocular. The preferred routes of administration are oral and intravenous.

Alternatively, one may administer the compound in a local rather than systemic manner, for example, via injection of the compound into a tumor as a depot or sustained release formulation.

### Compositions/Formulations

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable excipients that facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable excipients well-known in the art. Such excipients enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are normally provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be, added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

The compounds of this invention may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, carbonate, lactate, tartrate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), calcium hydroxide (Ca(OH)₂), etc.).

The compositions containing the compound(s) of the invention can be administered for prophylactic or therapeutic treatment. In therapeutic applications, the compositions are administered to a patient suffering from an progressive cancer in an amount sufficient to cure or at least partially arrest the growth or spread of the cancer. An amount adequate to accomplish this is defined as “therapeutically effective amount or dose.” Amounts effective for this use will depend on the severity and course of the cancer, previous therapy, the patient's health status and response to the drugs, and the judgment of the treating physician.

In prophylactic applications, compositions containing the compounds of the invention are administered to a patient who is either in remission or whose cancer is not progressing at a dangerous rate. The idea is that, while the cancer may not be eliminated, its progress can be arrested to a sufficient degree to maintain a reasonable quality of life for the patient. Such an amount is defined to be a “prophylactically effective amount or dose.” As above, the precise amounts again depend on the patient's state of health, weight, and the like.

As the patient's condition improves, the dosage or the frequency of administration, or both, can be reduced, as a function of the symptoms, to a level at which the improved condition is retained. When the symptoms have been alleviated to the desired level, treatment can cease. Patients can, however, require intermittent treatment on a long-term basis upon any recurrence of the disease symptoms.

### Dosage

According to this invention, a therapeutically effective amount of one or more of the compounds of this invention is administered to an organism suffering from a cancer. The proper dosage will depend on the severity and course of the infection, previous therapy, the patient's general health status, his or her response to the drugs, the therapeutic index of the drug, etc., all of which are within the knowledge, expertise and judgment of the treating physician.

In general, a suitable effective dose of the compound of the invention will be in the range of 0.1 to 10000 milligram (mg) per recipient per day, preferably in the range of 20 to 2000 mg per day. The desired dosage is preferably presented in one, two, three, four or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 2.0 mg/kg to 250 mg/kg of patient body weight, between about one to four times per day.

Once improvement of the patient's condition is observed, a maintenance dose may be administered if desired by the treating physician. The dosage, frequency, or both, can be reduced as a function of the patient's response to a level at which the improvement persists. When the symptoms have been alleviated to the desired level, treatment may be ceased although some patients may require intermittant treatment on a long-term basis should flare-ups of the symptoms reoccur.

Compound 1 or similar polymer platinum compound as described herein can be administered to a patient alone or in combination with other anticancer compounds and/or radiation to effect treatment.

Anticancer compounds that could be used with the platinum compounds of this invention include, but are not limited to: Acarbazine, Aldesleukin, Altretamine, Asparaginase, Bevacizumab, Bicalutamide, Bleomycin, Busulfan, Capecitabine, Carboplatin, Carmustine, Cetuximab, Chlorambucil, Cisplatin, Cladribine, Cyclophosphamide, Cytarabine, Dacarbazine, Depsipeptide Diethylstilbestrol, Doxorubicin, Dutasteride, Ethinyl, Erlotinib, Estradiol, Etoposide, Erythromycin, Finasteride, 5-Fluorouracil, 5-Fluorouracil plus leucovorin, Gefitinib, Gemcitabine, Goserelin, hexamethylmelamine, Ifosfamide, Interferon alfa-2b, Irinotecan, Leuprolide, Melphalan, Mitomycin, Mitotane, Mitoxantrone, Nilutamide, Ofloxacin, Oxaliplatin, Paclitaxel, Panitumumab, Pentastatin, Pemetrexed, Phenylbutyrate, Pipobroman, Plicamycin, Prednisone, Procarbazine, Streptozocin, Tamoxifen, Tamsulosin, Teniposide, Terazosin, Toptecan, Vinblastine, Vincristine, Vinorelbine, and Vorinostat.

Compound 1 or similar polymer platinum compound as described herein can also be used in neoadjuvant chemotherapy, adjuvant chemotherapy, or palliative chemotherapy.

### EXAMPLES

The examples provided herein are not intended nor should they be construed as limiting the scope of this invention in any manner whatsoever. In addition, where structures are presented relating to polymeric compounds of this invention, it is understood that the structure show is for illustrative purposes only and is not intended, nor is it to be construed, as limiting the scope of this invention in any manner. For instance, the compound of Example 25 is shown as comprising a poly(N-(2-hydroxypropyl)acrylamide-co-acrylamide) backbone polymer, an oligopeptide linker, 1R,2R-diaminocyclohexane as the stable bidentate amine ligand and several different N,O and N,N chelates coordinating the Pt species to the linker, each of these components can be replaced as described and claimed herein to provide other molecules of this invention. Furthermore, the number of Pt chelates shown is not intended, nor is it to be construed, to relate to the actual number or percentage of Pt species in the molecule as actually synthesized. In fact, as is described below, the amount of Pt per gram of compound of example 25 averages 37% more than that which would be expected if the linkers each only carried the one terminal Pt complex. Of course, depending on the synthetic method and amounts of reactants selected, this percentage is also controllably variable to give from 0% to 95% Pt content in excess of that calculated on a one linker/one Pt complex basis. Additionally, as will be apparent to those skilled in the art based on the disclosure and discussion herein, the specific structure of each linker-Pt chelate need not be exactly or only those shown; other N,O and N,N chelates are possible and all such chelates are within the scope of this invention.

### Example 1

### Preparation of the MAGGOH

A 3-neck, 2 L r.b. flask covered in Al-foil, equipped with a thermometer, mechanical stirring, and an addition funnel was charged with 600 mL of 5 N NaOH.7 An ice bath (−10° C.) was applied, and the reaction temperature was lowered to 0-5° C. While cooling, 150 g of Gly-Gly was added and stirred until dissolved. The pH was adjusted to 10.59 with 5N NaOH. Methacryloyl chloride (107.9 g, 1.032 mol, 100 mL) was cannulated into a dry addition funnel. With very vigorous stirring, the MA-Cl was added at a rate so that the temperature remained between 0-5° C. (ca. 1 h).

After all the MA-Cl had been added the pH was adjusted to 9-10, and the reaction mixture was stirred vigorously for 1.5-2 h at 0-5° C. A pH probe was inserted, and the pH was lowered to 1.5 with 6 N HCl (ca. 100-150 mL). Upon acidification copious amounts of a white precipitate (MA-GlyGly-OH) formed. The stirrer was removed, and the reaction mixture was placed at 4° C. overnight 14 (12-20 h).

The precipitate was collected by vacuum filtration on a 2 L medium glass frit and washed twice with 1 bed volume of cold water and twice with 1 bed volume of acetone. The filter cake was dried under a rubber dam for 20 min then at high vacuum overnight to give 150-170 g of MA-GlyGly-OH as white crystals and powder.

The crude MA-GlyGly-OH was transferred to 2 L media bottle wrapped in Al-foil. A magnetic stir bar, 100 mg of MEHQ, and 1.1 of abs. EtOH were added and brought to reflux. Once a solution was achieved, it was hot filtered through coarse filter paper. A screw cap was loosely applied and the solution allowed to cool to ambient temperature. Afterwards, it was placed at 4° C. for 12-20 h.

The crystals that formed were collected by vacuum filtration with a 2 L coarse glass frit, washed twice with 1-2 bed volumes of cold EtOH, and twice with one bed volume of cold acetone. The filter cake was dried under a rubber dam for several hours then at high vacuum for 12-20 h to give 153-170 g (75-82%) of MA-GlyGly-OH as a dry white solid, with <1% EtOH by 1H NMR spectroscopy.

### Example 2

### Preparation of the TFA-G-Ame

Diethylaminomalonate HCl (740.78 g, 3.50 mol) is placed into a 4 L Erlenmeyer flask equipped with a magnetic stir bar. A slurry of NaHCO₃ (325.00 g, 3.85 mol) in 1500 mL of water is added slowly. Upon reaction of the NaHCO₃ with the CH₂Cl hydrochloride salt, a gas evolves, and all the NaHCO₃ goes into solution. ₂ (1000 mL) is added after gas evolution has ceased. The resulting two layer mixture is vigorously stirred for 15 min, and the layers are separated using a 4 L separatory funnel. The organic layer is separated, 500 mL of CH₂Cl₂ is added, and the mixture is stirred as above. The organic layer is drawn off, combined with the previous one, then dried with 50 g of anhydrous Na₂SO₄ The mixture is slowly stirred for 2 min then allowed to stand for 10 min. The Na₂SO₄ is removed by gravity filtration using a 14 cm funnel (top I.D. 14.2 cm) with 24 cm medium filter paper. The volume of the Na₂SO₄ filtrate is reduced in vacuo to 600 mL.

The Na₂SO₄ solution is transferred to an oven dried 3 L 3 necked round bottomed (r.b.) flask equipped with a mechanical stirrer, thermometer, a 1 L additional funnel, and a positive pressure of argon. The t-BOC-Gly-OH (569.34 g, 3.25 mol) is added, and the resulting mixture is stirred and cooled to 10° C. using salt/ice bath. Once well stirred and at <10° C., DCC (670.57 g, 3.25 mol in 400 mL of CH₂Cl₂) is added via the addition funnel to the vigorously stirred diethylaminomalonate/t-BOC-Gly-OH mixture at a rate to maintain the temperature of the reaction mixture below 25° C. The DCC addition is typically complete with ca.2.5 h during which time a large amount of white precipitate (i.e. DCU) appears. After all of DCC is added, the mixture is stirred for 45 min<5° C. The ice bath is removed, the reaction mixture is stirred for additional 4 h at ambient temperature, then allowed to set overnight at ambient temperature (16-20 h).

The white DCU precipitate is removed by vacuum filtration with a 17 cm Buchner funnel fitted with 15 cm medium filter paper. The filter cake is washed with 100 mL of CH₂Cl₂, and the filtrates are combined in a 2 L 1-necked r.b. flask. The solvent is removed in vacuo yielding a light yellow, crystalline material which is dried for at least 6 h at <1 torr to give by tare 881.8 g (81.6%) of t BOC-Gly-Ame. This product is analyzed by 1H NMR spectroscopy for identity and purity.

### Example 3

### Preparation of the MA-GGG-Ame from MA-GG-OH

A dry 22 L round bottomed. flask equipped with a 6″ flange a 6″ head, mechanical stirring, and a thermocouple was put under an argon atmosphere and charged with MA-GlyGly-OH (500 g, 2.498 mol) and EDC (526.7 g, 2.748 mol). After an inert atmosphere was established, MeCN (6 L) was added at ambient temperature. Stirring was established, and as soon as a uniform white slurry formed, a previously prepared solution of TEA (556.0 g, 5.495 mol) and TFA Gly-Ame in MeCN 3.4 L was quickly added (<5 min). A slight exotherm was observed (to 40-45° C.). After stirring for at least 12 h, aliquots of the reaction mixture were analyzed by 1H NMR spectroscopy. When the reaction was >95% complete, the mixture was cooled to <0° C., filtered onto Sharkskin filter paper in an 18″ Buchner funnel, and washed with cold MeCN (0.5 L). The filter cake was partially dried overnight under a rubber dam to give 2.132 kg.

The filter cake was dissolved in a warm solution of 1:1 MeCN/H20 (6 L, (50-60° C.) in a 22 L round bottomed flask equipped with a heating mantle and mechanical stirring. The solution was cooled to <−10° C. without stirring to afford white granular MA-GGG-Ame. The granules were collected, without stirring, by vacuum filtration onto Sharkskin filter paper in a 18″ Buchner funnel and washed with cold MeCN/H₂O (1:1, 1 L, <−10° C.). The material was dried under a rubber dam for a few hours before performing the final recrystallization.

The final recrystallization was performed as above except that the homogenous warm solution was pressure filtered (<30 psi) through coarse filter paper (Fisher P8). After cooling at <−10° C. for 12 h the granular MA-GlyGlyGly-Ame product was collected as above, washed twice with cold MeCN/H₂O (1:1, 1-2 L, <−10° C.), twice with cold MeCN, and dried in vacuo at ambient temperature then at 35-40° C. for >72 h to give 725.0 g (70%) of MA-GlyGlyGly-Ame as a white powder: mp 174-180° C. (dec.); ¹H NMR (300 MHz, DMSO-□6) 08.79 (d, 1, —NHCHC02Et), 8.19 (t, 1, —NHCHr), 8.19 (t, 2, —NHCH2-), 5.74 (s, 1, ═CH2), 5.38 (s, 1, ═CH2), 5.07 (d, 1, —NHCHC02Et), 4.20-4.14 (m, 4, —NHCHC02CH2CH3), 3.83 (d, 2, —NHCH2-), 3.77-3.73 (m, 4, —NHCH2-), 1.88 (s, 3, —CH3), 1.20 (t, 6, —NHCHC02CH2CH3); ¹³C NMR (75 MHz, DMSO-d6) 166.8, 166.60, 166.56, 165.2, 163.73, 136.9, 117.3, 59.3, 53.6, 40.0, 39.4, 38.9, 16.0, and 11.3.

### Example 4

### Preparation of MA-GGG-OH

A 22 L round bottom flask equipped with a 6″ flange, mechanical stirring set-up, thermocouple, Al-foil, and an ice-salt bath was charged with GlyGlyGly (1.500 kg, 7.929 mol) and 4 L DI water. While stirring NaOH (475.76 g, 11.89 mol) dissolved in 2 L of DI water was added until the pH was 9-10.5, the GlyGlyGly had dissolved, and the mixture was at 0-5° C.

To this vigorously stirring reaction MA-Cl (0.8289 kg, 7.929 mol, 0.774 L) was added by peristaltic pump at a rate so that the temperature remained below <5° C. (˜2 h). The pH was monitored and maintained at pH 9-10.5 with 5 N NaOH during the MA-Cl addition. Afterwards, the reaction mixture was stirred at 0-5° C. for an additional 3 h then left stirring overnight.

After warming to ambient temperature overnight, the reaction mixture was cooled to ˜0° C. and acidified to pH 1.5 with 35%. nitric acid 12 (1.65 L). While acidifying, copious amounts of white precipitate formed. The thick, white slurry was stirred at 0-10° C. for 30 min then filtered using a 18″ stainless steel Buchner funnel equipped with two layers of Sharkskin filter paper. After DI water was used to complete the transfer the filter cake was triturated with 4 L of cold MeCN and dried overnight under a rubber dam to give 3.276 kg of product containing 48% H₂0 and 9.6% MeCN by weight. This was dried at 45-50° C. under high vacuum for ˜70 h to give a white mass of 2.18 kg with 37% H₂O and trace, if any, MeCN. While the water content was reduced, it was still above the acceptable content of <5%. Therefore, the material was triturated with 4 L of MeCN in a 22 L reactor for 1.5 h at ambient temperature. The product was collected by filtration as before, washed with 1 bed volume of MeCN, and partially dried under a rubber dam (<10 torr) overnight to give 2.052 kg of white solid containing 15% H20 and 13% MeCN. This was vacuum dried at 45-50° C. and <1 torr for 2 days to give 1.492 kg (73%) of a MA-GlyGlyGly-OH as a white solid with 2.8% H₂O (w/w) and no detectable MeCN. No further purification was performed before using this material to prepare MAGlyGlyGly-Ame.

### Example 5

### Preparation of MA-GGG-Ame from MA-GGG-OH

To a magnetically stirred solution of Ame-HCl (181.0 g, 0.8552 mol) in 300 mL of DI water in a 2 L beaker, NaHCO₃ (107.75, 1.2818 mol) was added slowly. After the CO₂ evolution ceased, 0.3 L of CH₂Cl₂ was added. The mixture was well agitated for 15 min then the organic layer was drawn off. The aqueous layer was extracted again with 0.2 L of CH₂Cl₂. The organic layers were combined and dried with approximately 25 g of anhydrous MgS04. After stirring for at least 15 min the MgS0₄ was removed by filtration. The filtrate was stripped in vacuo to give the Arne free base.

A 5 L round bottomed flask equipped with mechanical stirring, argon inlet and thermocouple was charged with MA-GGG-OH (183.3 g, 0.7126 mol), HOBt (14.44 g, 0.1068 mol), Arne free base (from previous step) dissolved in 0.5 L MeCN, and 2 L of dry MeCN. After stirring for 15 min, EDC-HCl (150.26, 0.7838 mol) was added. After stirring the resulting slurry overnight at ambient temperature and after an aliquot showed the reaction was >90% complete, the mixture was cooled to <0° C. and filtered through a 4 L glass frit with coarse filter paper. The filter cake was washed with 1 bed volume of cold MeCN. The wet filter cake was stirred with 1 L each of MeCN and H₂O in a 3 L beaker. Upon heating to 70° C., a clear solution formed. This was filtered by pumping the solution through an inline filter. 10 The filtrate was cooled to near ambient then placed in the freezer overnight. The product was collected by filtration using a 4 L coarse glass frit fitted with coarse filter paper to give 395 g of wet product. This was dried at <1 torr and 40° C. for ˜48 h to give 213 g (72%) of MA-GGG-Ame.

### Example 6

### Preparation of the Poly(HPMA)-GGG-Ame Polymer

### A. Polymer Preparation in Ethanol

An oven dried 5 L media bottle equipped with a magnetic stir bar, a 3 port injection cap, a Teflon tubing sparger, and under an Ar_{(g)} flow was charged with HPMA (150 g, 1.05 mol, 14.0 eq), EtOH (1900 mL), MA-GGG-Ame (31.08 g, 0.075 mmol, 1 eq), and V-65 (5.068 g, 0.02 mmol, 0.27 eq). The solution was 12% (w/w) monomers/solvent and 0.3% V-65 (w/w). The media bottle was wrapped in Al foil, and the mixture was purged/sparged with Ar for 1 h while maintaining vigorous stirring. Since oxygen is an excellent polymerization inhibitor, each reaction is sparged with UHP grade argon and sealed to prevent its presence. No condenser was used to recover the EtOH vapor lost, if any, during sparging. The heterogeneous mixture (the mixture was heterogeneous due to the insolubility of the MA GGG-Ame monomer (10-30% insoluble material)) was sealed from the Ar flow and heated at 50° C. in a water bath with stirring.

After 24 h, the nearly homogeneous reaction mixture was removed from the water bath and allowed to cool to room temperature. After venting, the reaction mixture was filtered through coarse filter paper to remove the minor amount of particulates within the mixture. The homogeneous opaque solution was split into 4 equal volumes (˜500 mL each) and added to 4 separate 5 L media bottles each containing vigorously stirring EtOAc (3 L each). Precipitation was immediate, and the precipitated mixture was allowed to stir for 1 h. The polymer was collected via centrifugation at 3800 rpm for 10 min at 10° C. The polymer precipitate was washed with EtOAc (1×1-2 bed volumes), Et₂O (2×1-2 bed volumes), and centrifuged after each washing. The precipitate was dried in vacuo to afford 143 g (79% yield) of white polymer. There was ˜6% solvent (EtOH, DMF, and EtOAc) impurity remaining within the polymer even after drying overnight under high vacuum at 40° C. ¹H NMR (300 MHz, D₂O) δ: 0.85-1.10, RCH₂C(CH₃)C(O)R (br s, 3H), 1.10-1.30, RNHCH₂CH(OH)CH₃, (br d, 3H), 1.30-1.40, RC(O)OCH₂CH₃, (br t, 3H), 1.65-2.05, RCH₂C(CH₃)C(O)R (2 broad peaks, 4H), 3.0-3.3, RNHCH₂CH(OH)CH₃ (two broad peaks, 2H), 3.85-4.0, RNHCH₂CH(OH)CH₃ (br s, 1H), 3.95-4.1, RNHCH₂C(O)R (br s, 6H), 4.25-4.40, ROCH₂CH₃ (br m, 4H). The amide and alcoholic protons have exchanged for deuterons as indicated by the HOD peak (sharp singlet) at 4.80 ppm.

### B. Polymer Preparation in DMF

An oven dried 5 L media bottle equipped with a magnetic stir bar, a 3 port injection cap, a Teflon tubing sparger, and under an Ar_{(g)} flow was charged with HPMA (149.7 g, 1.05 mol, 13.0 eq), DMF (1620 mL), MA-GGG-Ame (33.27 g, 0.08 mmol, 1 eq), and V-65 (3.424 g, 0.01 mmol, 0.17 eq). The solution was 12% (w/w) monomers/solvent and 0.2% V-65 (w/w). The media bottle was wrapped in Al foil, and the mixture was purged/sparged with Ar for 1 h while maintaining vigorous stirring. The heterogeneous mixture (the mixture was heterogeneous due to the insolubility of the MA-GGG-Ame monomer (˜5% insoluble material)) was sealed from the Ar flow and heated at 50° C. in a water bath with stirring.

After 24 h, the homogeneous reaction mixture was removed from the water bath and allowed to cool to room temperature. After venting, the homogeneous solution was split into 3 volumes (500 mL, 500 mL, and 700 mL) and added with vigorous stirring to two 5 L media bottles containing EtOAc (3 L) and one 10 L media bottle containing EtOAc (4 L), respectively. Precipitation was immediate, and the precipitated mixture was allowed to stir for 1 h. The polymer was collected via centrifugation at 3800 rpm for 10 min at 10° C. The polymer precipitate was washed with EtOAc (1×1-2 bed volumes), Et₂O (2×1-2 bed volumes), and centrifuged after each washing. The polymer was dried overnight under high vacuum at 40° C.

The ¹H NMR spectrum indicated greater than 10% DMF by weight, thus the polymer was redissolved with EtOH (1.5 L). Once in solution (˜2 h), it was split into 4 batches of ˜500 mL each. Each solution was gravity filtered directly into 4 separate 5 L media bottles containing vigorously stirring EtOAc (3 L each). Here, coarse filter paper was used to remove any undissolved particulates as well as to insure slow addition of the DMF solution. Precipitation was immediate and the precipitated mixture was allowed to stir for 1 h. The polymer was collected via centrifugation at 3800 rpm for 10 min at 10° C. The polymer precipitate was washed with EtOAc (1×1-2 bed volumes), Et₂O (2×1-2 bed volumes), and centrifuged after each washing. The precipitate was dried in vacuo to afford 131 g (71% yield) of white polymer. There was ˜8% solvent (EtOH, DMF, and EtOAc) impurity remaining within the polymer even after drying overnight under high vacuum at 40° C. ¹H NMR (300 MHz, D₂O) □: 0.85-1.10, RCH₂C(CH₃)C(O)R (br s, 3H), 1.10-1.30, RNHCH₂CH(OH)CH₃, (br d, 3H), 1.30-1.40, RC(O)OCH₂CH₃, (br t, 3H), 1.65-2.05, RCH₂C(CH₃)C(O)R (2 broad peaks, 4H), 3.0-3.3, RNHCH₂CH(OH)CH₃ (two broad peaks, 2H), 3.85-4.0, RNHCH₂CH(OH)CH₃ (br s, 1H), 3.95-4.1, RNHCH₂C(O)R (br s, 6H), 4.25-4.40, ROCH₂CH₃ (br m, 4H). The amide and alcoholic protons have exchanged for deuterons as indicated by the HOD peak (sharp singlet) at 4.80 ppm.

### C. Alternative Polymer Preparation in DMF

An oven dried 1 L media bottle was equipped with a magnetic stir bar, a 3 port injection cap, and a Teflon tubing sparger. HPMA (50 g, 0.35 mol, 13.0 eq) and MA-GGG-Ame (11.13 g, 0.026 mol, 1.0 eq) were charged and then DMF (540 mL) was added. The reaction mixture was vigorously stirred and sparged with Ar for one hour. After one hour, sparging of Ar was stopped and AIBN (3.97 g, 0.024 mol, 0.9 eq) added. Again the reaction mixture was sparged for 10 to 15 minutes. This reaction mixture was placed in a water bath and was heated to 65° C. within 30 minutes. At this temperature the reaction mixture was allowed to stir for 16 hours. ¹H-NMR spectroscopy of aliquots at 12 hours shows 18% of HPMA and at 16 hours shows 13.8% of HPMA in the reaction mixture.

After 16 hours the homogeneous reaction mixture was taken out from the water bath and allowed to cool to room temperature. This reaction mixture was added slowly into a 4 L media bottle containing 3.33 L of vigorously stirring ethyl acetate forming a white solid. This precipitated polymer was collected by centrifugation and washed with ethyl acetate (3×2 bed volumes). The polymer was dried under high vacuum for 3 days gave 54.74 g (89.6%) product. ¹H NMR (300 MHz, DMSO-d₆) δ: 0.83, RCH₂C(CH₃)R (br s, 6H), 1.04, RCH(OH)CH₃ (br d, 3H), 1.22, RC(O)OCH₂CH₃ (br t, 3H), 1.40-2.30, RCH₂C(CH₃)C(O)R (2 broad peaks, 4H), 2.93, RNHCH₂CH(OH)CH₃ (br s, 2H), 3.69, RNHCH₂CH(OH)CH₃ (br s, 1H), 3.86, RNHCH₂C(O)R (br s, 6H), 4.30, ROCH₂CH₃ (br m, 4H), 4.71, RCH(OH)CH₃, (br s, 1H), 5.08, RNHCH(C(O))₂ (d, 1H), 6.60-7.70, RNHCH₂CH(OH)CH₃ (br multiplet, 1H), 7.98-8.47, linker NH (br multiplet, 3H), 8.83, RNHCH(C(O))₂, (br peak, 1H).

### Example 7

### Control of Molecular Weight in the Preparation of the poly(HPMA)-GGG-Ame Polymer

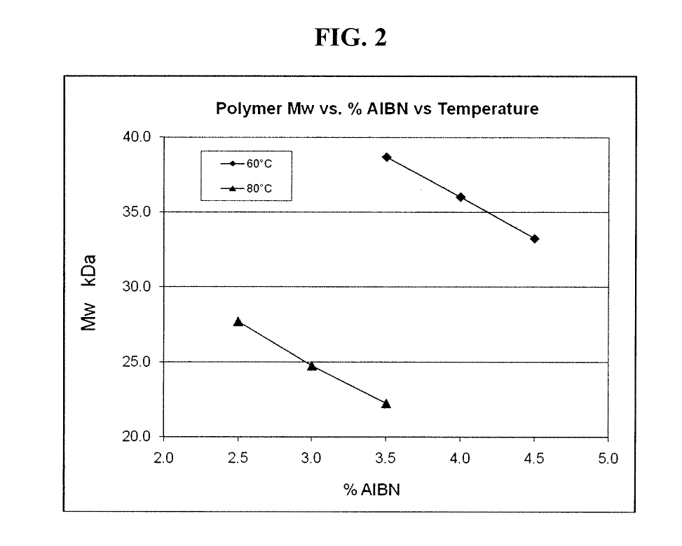

A 100 mL media bottle with magnetic stir-bar was oven-dried (120° C.), fitted with a S-port cap and allowed to cool under dry Ar. The HPMA monomer (5.000 g) and AME monomer (1.113 g) were added to the bottle, followed by dimethyl formamide (54 mL) and the mixture was stirred and sparged with Ar for 1 hour. The appropriate quantity of AIBN or V-65 (see table) was added to the reaction and it was sparged with Ar for 10 min. The reaction was then heated in an oil bath, reaching reaction temperature in 30-45 min., and it was maintained at reaction temperature for a further 18 hours. The reaction was then poured slowly into rapidly stirring ethyl acetate (330 mL), resulting in a white precipitate which was stirred for a further 5-10 min. The suspension was centrifuged (4200 rpm; 10 min.) and the supernatant was decanted. The precipitate was washed twice with ethyl acetate and once with diethyl ether by resuspending, centrifuging and decanting. The resulting material was dried under vacuum to give a white solid. AME contents were determined by ¹H NMR and Mw values determined by SEC. The reaction conditions and outcomes are summarized in the table below and in FIG. 2.

| Temp. | % AIBN | % Yield | M_{w} - kDa |
|---|---|---|---|
| 80° C. | 2.5 | 83 | 27.7 |
| | 3.0 | 81 | 24.8 |
| | 3.5 | 85 | 22.3 |
| 60° C. | 3.5 | 81 | 38.7 |
| | 4.0 | 82 | 36.0 |
| | 4.5 | 83 | 33.2 |
| Temp. | % V-65 | % Yield | M_{w} - kDa |
| 65° C. | 1.3 | 74 | 34.7 |
| | 1.6 | 73 | 28.2 |
| | 2.0 | 70 | 26.0 |

### Example 8

### Preparation of Platinating Solutions

### A. Preparation of cis-diamminediaquaplatinum (II) dinitrate (1a) Solution

A suspension of cisplatin (8.996 g, 29.98 mmol), AgNO₃ (9.959 g, 58.62 mmol), 3-5 drops of 5% HNO₃, and 190 mL of water were stirred overnight at about 23° C. in a foil-covered low actinic media bottle and then heated at 60-65° C. for 3.5 h. After cooling to ambient temperature, the mixture was filtered through a 0.22 μM filter to give a solution of 1a with a pH of approximately 2. A Pt and Ag analysis (ICP-OES) showed that it contained ca. 20,000-25,000 ppm of Pt and 4-14 ppm of Ag. Each preparation was analyzed for Pt, and, just prior to use, it was heated to 55° C. for 5 min then cooled to ambient temperature.

### B. Preparation of 1R,2R-DACH-diaquaplatinum (II) dinitrate (1b) Solution

A suspension of cis-1R,2R-diaminoccylohexane(DACH)dichloroplatinum (H) (2.99 g, 7.86 mmol), Ag(NO₃) (2.6137 g, 15.38 mmol), 2-3 drops of 5% HNO₃ and 56 mL of water were stirred overnight at about 23° C. in a foil-covered media bottle then heated at 60-65° C. for 3.5 h. After cooling to ambient temperature the mixture was filtered through a 0.22 μm filter. The pH of the solution was about 2. Pt analysis showed that it contained ca. 15,000-25,000 ppm of Pt. Each preparation was analyzed for Pt and just prior to use it was heated to 55° C. for 5 min then cooled to ambient temperature.

### C. Preparation of 1R,2R-DACH-diaquaplatinum (II) dinitrate (1b) Solution

A 250 mL media bottle containing a stir bar was wrapped in foil and charged with DACHPt(NO₃)₂ (13.7039 g, 31.63 mmol) and water (204 mL). 5% HNO3 (900 μL) was added, the vessel capped and the mixture stirred in a 70° C. water bath for 1 h. The platinating solution was filtered through a 0.22 μm filter and cooled to room temperature prior to use.

### Example 9

### Preparation of poly(HPMA)-GGG-Ama=PtDACH

A 500 mL media bottle containing a stir bar was charged with AP5195 (50.00 g, 25.045 mmol Ame) and water (280 mL). Upon dissolution of the polymer 2M NaOH (18 mL) was added to the stirring solution (approximately 60% of the calculated amount) and the pH rose to 12.6 where it was maintained for 30 min. The hazy solution was then taken to pH 7.4 by the slow addition of 5% HNO₃ (10.5 mL), and then filtered through a Milligard Opiticap XL2 device fitted with 1.2 μm and 0.5 μm membranes. The clean filtrate was filtered through a Steritop and then previously prepared platinating solution was added with vigorous stirring. The pH dropped to 4.8 and was raised to 5.2 using 2M NaOH. The solution was then stirred for 2 h with the pH held constant using a Mettler DL25 autotitrator loaded with 2M NaOH. The solution (600 mL in volume) was clear and bright yellow. The pH was adjusted to 7.4 using 2M NaOH and placed in a 38° C. water bath and pH held constant at 7.4 for 17 h. The clear, pale yellow solution was filtered through a Steritop and purified by TFF (5 permeates collected). It was made 200 mM in chloride and 80 mM in phosphate by addition of NaCl (5.8440 g, 100.00 mmol), NaH₂PO₄.H₂O (1.1039 g, 8.00 mmol) and Na₂HPO₄.7 H₂O (8.5782 g, 32.00 mmol). The solution was diluted to 500 mL (10.00 wt. %) and stirred. After dissolution of the salts pH was 7.1. This was raised to 7.4 and the solution filtered, and quickly heated to 38° C. in a 70° C. water bath. The vessel was then placed in a 38° C. water bath for 4.5 h with no stirring. The clear solution was filtered through a Steritop, purified by TFF (7 permeates collected) and lyophilized to yield a white solid (43.92 g, 75%). ¹⁹⁵Pt NMR (93:7 H₂O:D₂O): −2260, −2290 ppm.

### Example 10

### Synthesis of poly[(HPMA)-Ama=Pt(IV)(OH)₂DACH]

### a. Synthesis of poly[(HPMA)-Ame]

An oven-dried foil-wrapped 500 mL media bottle was evacuated, filled with argon and charged with diethyl N-methacroylaminomalonate (2.50 g, 10.3 mmol), HPMA (13.2 g, 92.5 mmol), V65 (0.39 g, 1.58 mmol) and absolute ethanol (200 mL). The solution was stirred with sparging argon for 90 min and then the vessel was sealed and placed in a 50° C. water bath, with gentle stirring, for 22 h. The reaction mixture was then cooled to room temperature and filtered into 1.5 L of stirring ethyl acetate, forming a white precipitate. This was stirred for 30 min and the solid material collected by centrifugation then dried in-vacuo to yield 9.52 g (61%) of the product as a white powder. SEC Mw 35.6 kDa, 0.555 mmol Ame/g polymer by ¹H NMR.

### b. Synthesis of poly[(HPMA)-Ama=Pt(IV)(OH)₂DACH]

A foil-wrapped 50 mL media bottle was charged with PtDACH(NO₃)₂ (2.00 g, 4.62 mmol), deionised water (27 mL) and 5% HNO₃ (4 drops). The mixture was stirred in a 70° C. water bath for 2 h, to yield a clear pale yellow solution of PtDACH(OH₂)₂, which was cooled to room temperature prior to use.

A 100 mL media bottle was charged with poly[(HPMA)-A e] (7.5 g, 4.16 mmol Ame) and deionized water (42 mL) with stirring to form a colorless solution of pH 4.5. The pH was raised to 12.6 by addition of 2M NaOH and the solution stirred at this pH for 30 min. 5% HNO3 was then added until pH 7.4 and the PtDACH(OH₂)₂ added, dropping the pH to 4.8. This was adjusted to 5.2 and the solution stirred in the range of pH 5.0-5.4 for 2 h. 50% H₂O₂ solution (1.4 mL) was then added and the reaction mixture stirred in a 65° C. water bath for 3 h. During this period the pH remained stable at 3.8. The solution was then brought to room temperature and filtered through a 0.22 μm membrane. The filtrate was purified by tangential flow filtration using a 5 kDa NMWCO membrane, and lyophilized to yield the product as a white powder (6.95 g, 93%). δ (Pt)=1486.

### Example 10

### Synthesis of poly[(HPMA)-Ama=Pt(IV)(OCOCH₃)₂DACH]

A 50 mL media bottle was charged with poly[(HPMA)-Ama=PtIV(OH)₂DACH] (1.00 g, 1.11 mmol OH groups) and DMF (15 mL). The mixture was stirred for 30 min to give a pale yellow solution, and was then charged with excess acetic anhydride (1.13 g, 11.1 mmol), with stirring in an 80° C. water bath for 2 h, forming a light orange solution. This was allowed to come to room temperature and then poured into 100 mL of stirring ethyl acetate, precipitating out an off-white solid. This was washed with ethyl ether (3×100 mL), isolated by centrifugation, and dried in-vacuo to yield the product as an off-white powder (1.00 g, 100%). d(Pt)=1761.

### Example 11

### Synthesis of poly[(HPMA)-Ama=Pt(IV)(OCOCF₃)₂DACH]

A 50 mL media bottle was charged with poly[(HPMA)-Ama=PtIV(OH)₂DACH] (1.00 g, 1.11 mmol OH groups) and DMF (15 mL). The mixture was stirred for 30 min to give a pale yellow solution, and was then charged with trifluoroacetic anhydride (0.23 g, 1.11 mmol), with stirring at room temperature for 2 h. The solution was poured into 100 mL of stirring ethyl acetate, precipitating out an off-white solid. This was washed with ethyl ether (3×100 mL), isolated by centrifugation, and dried in-vacuo to yield the product as an off-white powder (0.92 g, 92%). δ(Pt)=1514.

### Example 12

### Synthesis of poly[(HPMA)-Ama=Pt(IV)(OCOC₃H₇)₂DACH]

A 50 mL media bottle was charged with poly[(HPMA)-Ama=Pt(IV)(OH)₂DACH] (1.00 g, 1.11 mmol OH groups) and DMF (15 mL). The mixture was stirred for 30 min to give a pale yellow solution, and was then charged with excess n-butyric anhydride (1.76 g, 11.1 mmol), with stirring in an 80° C. water bath for 2 h, forming a light orange solution. This was allowed to come to room temperature and then poured into 100 mL of stirring ethyl acetate, precipitating out an off-white solid. This was washed with ethyl ether (3×100 mL), isolated by centrifugation, and dried in-vacuo to yield the product as an off-white powder (1.00 g, 100%). δ (Pt)=1762.

### Example 13

### Synthesis of poly[(HPMA)-Ama=Pt(IV)(OCOC₅H₁₁)₂DACH]

A 50 mL media bottle was charged with poly[(HPMA)-Ama=Pt(IV)(OH)₂DACH] (1.00 g, 1.11 mmol OH groups) and DMF (15 mL). The mixture was stirred for 30 min to give a pale yellow solution, and was then charged with excess hexanoic anhydride (2.38 g, 11.1 mmol), with stirring in an 80° C. water bath for 2 h, forming a light orange solution. This was allowed to come to room temperature and then poured into 100 mL of stirring ethyl acetate, precipitating out an off-white solid. This was washed with ethyl ether (3×100 mL), isolated by centrifugation, and dried in-vacuo to yield the product as an off-white powder (1.00 g, 100%). δ (Pt)=1761

### Example 14

### Synthesis of poly[(HPMA)-Ama=Pt(IV)(OCOC₂H₄CO₂H)₂DACH]

A 50 mL media bottle was charged with poly[(HPMA)-Ama=Pt(IV)(OH)₂DACH] (1.00 g, 1.11 mmol OH groups) and DMF (15 mL). The mixture was stirred for 30 min to give a pale yellow solution, and was then charged with excess succinic anhydride (1.11 g, 11.1 mmol), with stirring in an 80° C. water bath for 2 h, forming a light orange solution. This was allowed to come to room temperature and then poured into 100 mL of stirring ethyl acetate, precipitating out an off-white solid. This was washed with ethyl ether (3×100 mL), isolated by centrifugation, and dried in-vacuo to yield the product as an off-white powder (1.20 g, >100% due to DMF). δ (Pt)=1768.

### Example 15

### Synthesis of poly[(HPMA)-Ama=Pt(IV)(OCOPh)₂DACH]

A 50 mL media bottle was charged with poly[(HPMA)-Ama=Pt(IV)(OH)₂DACH] (1.00 g, 1.11 mmol OH groups) and DMF (15 mL). The mixture was stirred for 30 min to give a pale yellow solution, and was then charged with excess benzoic anhydride (2.51 g, 11.1 mmol), with stirring in an 80° C. water bath for 6 h, forming a light orange solution. This was allowed to come to room temperature and then poured into 100 mL of stirring ethyl acetate, precipitating out an off-white solid. This was washed with ethyl ether (3×100 mL), isolated by centrifugation, and dried in-vacuo to yield the product as an off-white powder. δ (Pt)=1753.

### Example 16

### Synthesis of poly[(HPMA)-Ama=Pt(IV)(OCOC₆H₄CO₂H)₂DACH]

A 20 mL vial was charged with poly[(HPMA)-Ama=Pt(IV)(OH)₂DACH] (0.50 g, 0.63 mmol OH groups) and DMF (6 mL). The mixture was stirred to form a solution and then charged with phthalic anhydride (93.0 mg, 0.63 mmol) with stirring at room temperature for 3 days. The solution was then poured into 30 mL of stirring ethyl acetate, precipitating out an off-white solid. This was washed with acetone (3×30 mL), isolated by centrifugation, and dried in-vacuo to yield product as an off-white powder (0.53 g, 100%). δ (Pt)=1778.

### Example 17

### Synthesis of poly[(HPMA)-Ama=Pt(IV)(OCOCH₂OCH₂CO₂H)₂DACH]

A 20 mL vial was charged with poly[(HPMA)-Ama=Pt(IV)(OH)₂DACH] (0.50 g, 0.63 mmol OH groups) and DMF (6 mL). The mixture was stirred to form a solution and then charged with diglycolic anhydride (0.73 g, 6.27 mmol) with stirring in an 80° C. water bath for 2 h. The solution was cooled to room temperature and then poured into 30 mL of stirring ethyl acetate, precipitating out a gummy orange solid. This was washed with acetone (3×30 mL), isolated by centrifugation, and dried in-vacuo to yield product as a yellowish powder. δ (Pt)=1773.

### Example 18

### Synthesis of poly[(HPMA)-Ama=Pt(IV)(OCOC₃H₆CO₂H)₂DACH]

A 20 mL vial was charged with poly[(HPMA)-Ama=Pt(IV)(OH)₂DACH] (0.50 g, 0.63 mmol OH groups) and DMF (6 mL). The mixture was stirred to form a solution and then charged with glutaric anhydride (0.72 g, 6.27 mmol) with stirring in an 80° C. water bath for 2 h. The solution was cooled to room temperature and then poured into 30 mL of stirring ethyl acetate, precipitating out an off-white solid. This was washed with acetone (3×30 mL), isolated by centrifugation, and dried in-vacuo to yield product as an off-white powder. δ (Pt)=1757.

### Example 19

### Synthesis of poly(HPMA-co-MAGG-AHVB12-co-MAGGG-Ama=PtDACH)

### a. Synthesis of MAGG-ONp

A dry, 3-neck, 2 L flask covered in aluminum foil and equipped with a thermometer, a mechanical stirrer and a pressure equalizing addition funnel with a septum under argon was charged with methacryloylglycylglycine (MAGG-OH) (96.0 g), p-nitrophenol (66.7 g) and dry DMF (992 mL) and cooled to −10 to −20° C. in an ice bath. While stirring vigorously, a solution of dicyclohexylcarbodiimide (94.0 g) in dry DMF (94 g) was added over 20 min. so that the temperature remained below −10° C. The pasty mixture was stirred at −10 to −20° C. for 4 hours, during which time it turned from brown to golden to yellow, and then at ambient temperature for a further 20 hours. The reaction mixture was vacuum filtered through a medium porosity glass fritted Buchner funnel with a Whatman GF/B glass microfiber filter and the filter cake was washed twice with 1-2 bed volumes of cold DMF. Crushed ice (3 g per mL DMF) was added to the filtrate and washings separately, and the resulting precipitates were filtered and washed with 1-2 bed volumes each of water, ethyl acetate, ethanol and diethyl ether, respectively, and then dried. ¹H NMR analysis revealed similar purities for the precipitates from each of the filtrate and washings, so they were all combined and recrystallized from 50% aqueous ethanol (15% wt/vol) and subsequently from acetonitrile (15% wt/vol) to give pale yellow crystals (85 g; 58% yield) of methacryloylglycylglycine p-nitrophenyl ester, MAGG-ONp.

### b. Synthesis of poly(HPMA-co-MAGG-ONp)

To an oven-dried 500 mL media bottle equipped with a magnetic stir-bar and three-port cap and under a flow of argon was added acetone (435 mL), MAGG-ONp (10.1 g) and N-(2-hydroxypropyl)-methacrylamide (HPMA) (31.26 g), and the bottle was wrapped in aluminum foil. The mixture was sparged with argon for 1 hour with vigorous stirring. A solution of AIBN (2.33 g) in acetone (20 mL) was added and the mixture was sparged with argon for 1 hour. The bottle was sealed from the argon flow and the reaction was heated at 50° C. for 48 hours. After cooling to room temperature, the resultant precipitate was filtered, washed with acetone (3×) and with ether (3×), and then dried under vacuum to afford poly(HPMA-co-MAGG-ONp) (32.03 g) as an off-white solid. 1H NMR analysis confirmed the structure of the polymer and HPLC analysis gave the ONp content as 0.577 mmoles ONp groups per gram of polymer. The molecular weight distribution was measured by SEC analysis; Mw=18.3 kDa, Mn=12.0 kDa.

### c. Synthesis of poly(HPMA-co-MAGG-AHVB12-co-MAGGG-Ame)

To an oven-dried 250 mL media bottle equipped with a magnetic stir-bar and septum cap and under a flow of argon was added dry DMSO (40 mL) and poly(HPMA-co-MAGG-ONp) (8.1 g). When the polymer had dissolved, triethylamine (0.8 g) was added, followed by a solution of aminohexyl-VB12 (1.32 g) in dry DMSO (80 mL) and then solid aminohexyl-VB12 (0.77 g). The mixture was stirred under argon for 1.5 hours, at which time HPLC analysis revealed that 38% of the available ONp groups had been reacted. The trifluoroacetate salt of diethyl glycylamidomalonate (2.60 g) was added, followed by more triethylamine (1.33 g). The mixture was stirred under argon for 3 hours, at which time HPLC analysis revealed that 100% of the available ONp groups had been reacted. The mixture was poured into rapidly stirring ethyl acetate (900 mL) and the resulting precipitate was stirred for 45 min., and then collected by centrifugation at 4800 rpm for 6 min. at 10° C. The precipitate was washed with ethyl acetate and with ether twice, and then dried under vacuum. The material was dissolved in ethanol (60 mL) and Bio Rex MSZ 501D resin (50 g), which had been pre-washed with ethanol, was added and the mixture was shaken for 2.5 hrs. The resin was filtered and washed with ethanol (200 mL) until the washings were colorless. The filtrate and washings were combined and the solution concentrated to 100 mL on a rotary evaporator. The solution was poured into rapidly stirring ethyl acetate (600 mL), the resulting precipitate was stirred for 45 min., and then collected by centrifugation at 4800 rpm for 6 min. at 10° C. The precipitate was washed with ethyl acetate and with ether twice, and then dried under vacuum to afford poly(HPMA-co-MAGG-AHVB12-co-MAGGG-Ame) (9.3 g) as a red solid. ¹H NMR analysis confirmed the structure of the polymer and its molecular weight distribution was measured by SEC analysis; Mw=21.0 kDa, Mn=14.5 kDa.

### d. Synthesis of poly(HPMA-co-MAGG-AHVB12-co-MAGGG-Ama=PtDACH)

A 20 mL vial containing a stir bar was charged with platinum DACH dinitrate (1.3445 g) and water (11.6 mL). 5% Nitric acid (0.1 mL) was added, the vial was capped, wrapped in foil and the mixture stirred in a water bath at 70° C. for 1 hour. This platinating solution was cooled to room temperature prior to use. A 100 mL media bottle containing a stir bar was charged with poly(HPMA-co-MAGG-AHVB12-co-MAGGG-Ame) (6.00 g) and water (34 mL). Upon dissolution of the polymer, the pH of the solution was adjusted from 4.3 to 12.6 by the addition of 2 M sodium hydroxide (˜1.5 mL) and the clear, dark red solution was stirred for 30 min., maintaining the pH at 12.5-12.6 with 2 M NaOH. The solution was then adjusted to pH 7.4 by the slow addition of 5% nitric acid and then filtered through a 0.22 μm Steritop filter device. The platinating solution was then added with vigorous stirring, resulting in the pH dropping to 5.2. The flask was wrapped in aluminum foil and the solution stirred for 2 hours with the pH held constant at 5.2 using a Mettler DL25 autotitrator loaded with 0.6 M NaOH. The pH was adjusted to 7.4 using 2 M NaOH and the solution was then stirred for 17 hours in a 38° C. water bath and pH held constant at 7.4. The clear, dark red solution was filtered through a Steritop filter and diafiltered with 5 volumes of water through a 5 kDa MWCO 0.1 m2 tangential flow filtration (TFF) membrane. To the diafiltered solution was added sodium chloride (0.7013 g), sodium dihydrogen phosphate monohydrate (0.1325 g) and disodium hydrogen phosphate heptahydrate (1.0294 g). The solution pH was adjusted to 7.4 and the volume to 60 mL, then it was quickly heated to 38° C. and maintained at 38° C. for 5 hours. The solution was cooled, filtered through a Steritop filter, then subjected to TFF (7 volumes of water), and lyophilized to yield a red solid (6.2930 g).

### Example 20

### Determination of Tumor Growth Inhibition by a Vitamin B12 Derivative of Compound 1

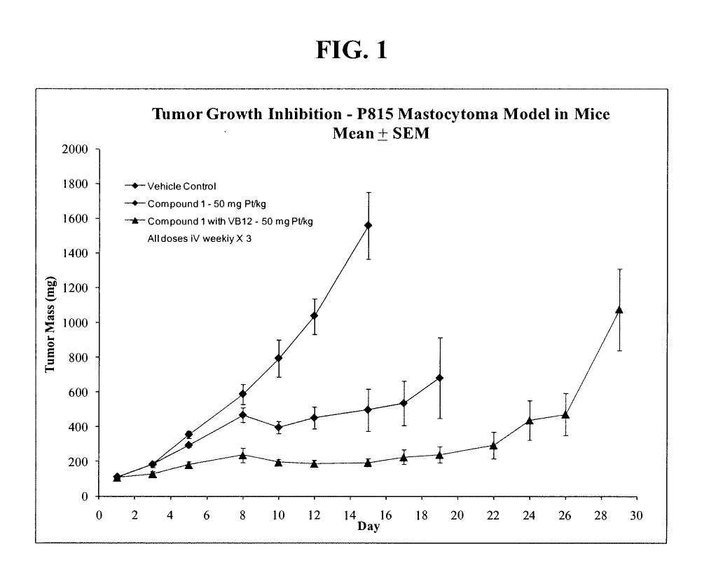

DBA/2 mice were innoculated with 2×106 P815 cells s/c. Mice were maintained on a diet deficient of folate and vitamin B12 throughout the study. Five days after inoculation, tumor sizes were measured and mice were weighed and placed into groups of 8. Animals were then treated with a) Compound 1 at a dose of 50 mg Pt/kg, b) Compound 1 formulated with vitamin B12 at a dose of 50 mg Pt/kg, or c) vehicle control by IV injection on treatment days 1, 8 and 15. Mice were given injections based on weight such that 20 g=200 μl injection. Tumor sizes and body weights were recorded 3 times per week until tumor weights had reached 2 grams, or until mice had died. As shown in FIG. 1, the group of animals treated with Compound 1 formulated with vitamin B12 exhibited more prolonged tumor growth inhibition than those treated with compound 1 with no vitamin B12 in the formulation. This provides good evidence that using vitamin B12 to target tumors can increase efficacy of the drug.

### Example 21

### Determination of the Relative Toxicity of Compound 1 Made by Two Different Processes

Female C57BL/6 mice were randomized into 7 groups of 5 mice each. Each mouse was identified by ear notch and followed individually throughout the study. Animals were observed and weighed prior to dosing for establishment of dosing volumes. All mice were dosed by a single IP injection with either a) Compound 1 made by the processes described herein, b) Compound 1 made by the process described in U.S. Pat. No. 7,166,733, or c) vehicle control. Three doses of each compound were tested: 75 mg Pt/kg, 100 mg Pt/kg and 150 mg Pt/kg. Test compounds were dissolved in isotonic glucose and administered in a volume of 0.4 mL per 20 g body weight. Gross observations and body weights were recorded daily for 14 days. Percent body weight change for each animal on each day was calculated as the percent change from their body weight recorded on day 1 just prior to injection of compounds.

No individual animal lost more than 10% of its body weight and no group exhibited more than 5.4% mean maximum body weight loss indicating that none of the test articles was toxic at the doses administered in this study. No treatment group was significantly different than any other with regard to mean percent body weight change. The study design called for dosing on days 0, 7 and 14. No further doses were given. As is usually the case in tumor models when some measurable tumor mass remains at the end of dosing, tumor regrowth occurs following cessation of dosing which is not the result of the development of resistance.

These data show that Compound 1 made by the processes described herein has similar toxicity to that of Compound 1 made by the old process.

Thus, the present invention provides a number of new processes for the preparation of HPMA polymer platinum complexes and several new HPMA polymer platinum complex compounds expected to be useful in the treatment of solid tumor cancers.

Although certain embodiments and examples have been used to describe the present invention, it will be apparent to those skilled in the art that changes in the embodiments and examples shown may be made without departing from the scope and spirit of this invention.

What is claimed:

1. A compound of formula 4: 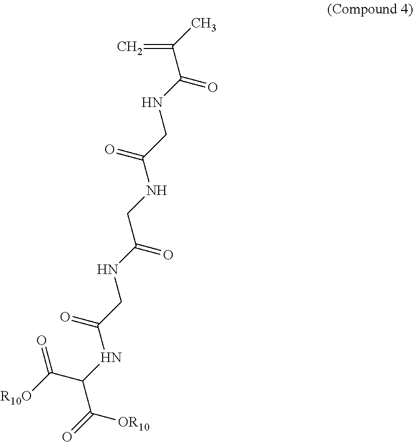
wherein R₁₀ is C₁-C₆ alkyl.

2. A method of manufacture comprising contacting a compound of formula 4: 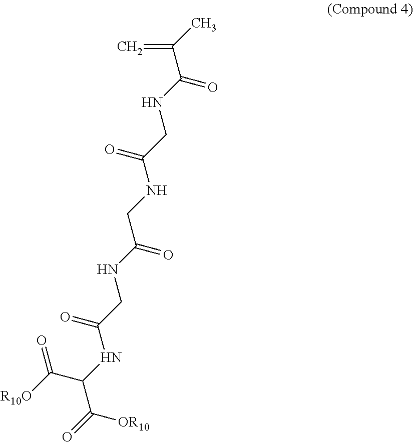
wherein R₁₀ is C₁-C₆ alkyl
with a compound of formula 3: 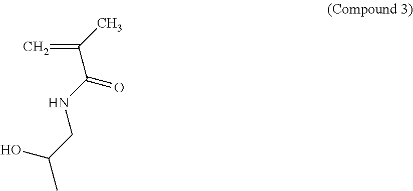
and a free radical initiator to provide a polymer of formula 2: 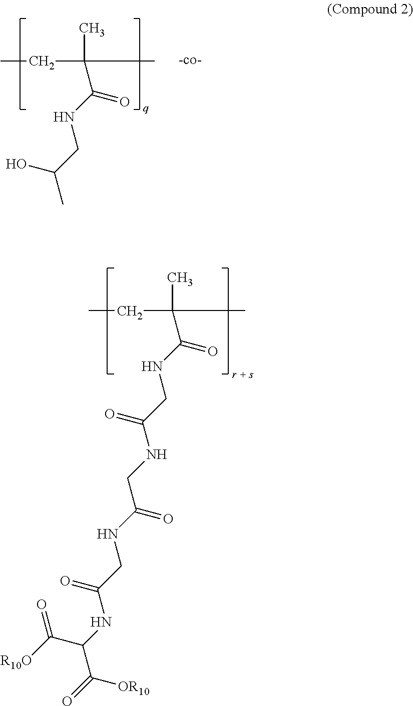
wherein
‘co’ represents binding of the individual monomer units to form a random copolymer.
q, r, and s represent the molar percentage of the monomer units in the polymer;
q is ≧50 and ≦95;
r+s is ≧5 and ≦50;

3. A polymer of formula 1b: 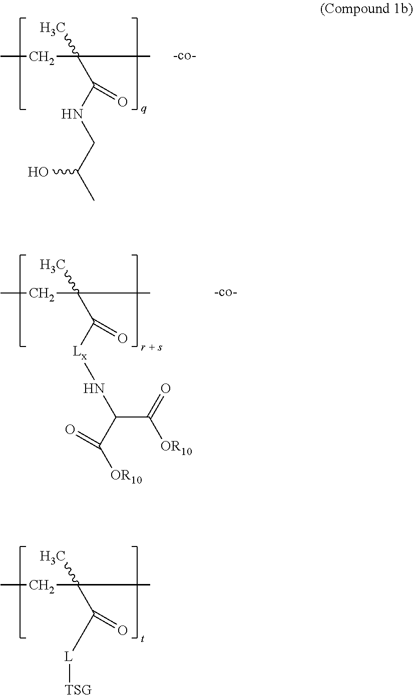
wherein
‘co’ represents binding of the individual monomer units to form a random copolymer.
q, r, s, and t represent the molar percentage of the monomer units;
q+r+s+t=100;
q is ≧50 and ≦95;
r+s is ≧5 and ≦50;
t is ≧0 and ≦30;
R₁₀ is C₁-C₆ alkyl or hydrogen;
TSG is a tumor seeking group that is capable of targeting cancerous tumor selected from the group of a monoclonal antibody, an antibody fragment, a peptide comprising 2-50 amino acids, a protein, a steroid, a somatostatin analog, a lectin, a folic acid or its derivatives and analogs, vitamin B12, biotin, porphyrin, an essential fatty acid, a bioreductive molecule and a polyanionic polysaccharide; and
L and Lₓ are linkers that are independently a covalent bond, or an amino acid, or a peptide comprising 2-50 amino acids, or a straight or branched C₁-C₁₀ alkylene chain; or a combination of these components;
or a salt or ester thereof.

4. The polymer of claim 3 of formula 6: 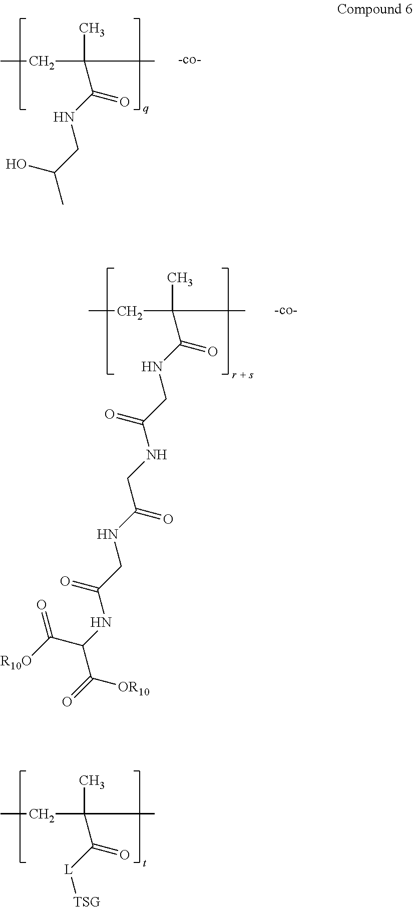
wherein t is ≧1 and ≦30.

5. A compound of formula 7: 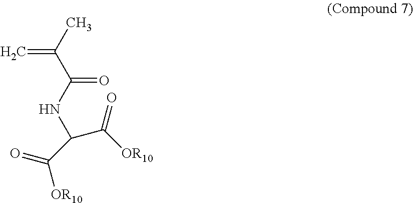
wherein R₁₀ is C₁-C₆ alkyl.

6. The compound of claim 1 or 5, wherein R₁₀ is methyl or ethyl.

7. The polymer of claim 3 of formula 8: 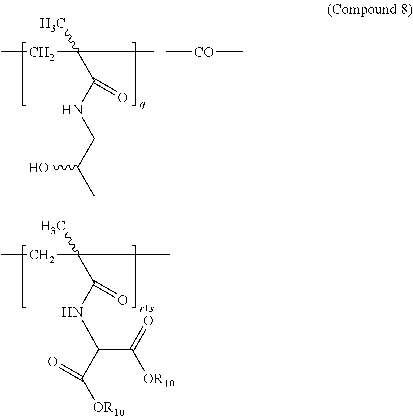
wherein
q is ≧50 and ≦95
r+s is ≧5 and ≦50.

8. A method of manufacture comprising contacting a compound of formula 7: 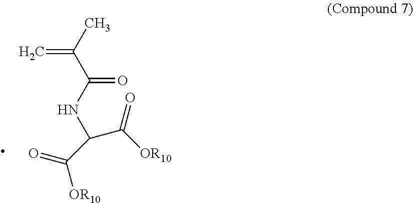
wherein R₁₀ is C₁-C₆ alkyl
with a compound of formula 3: 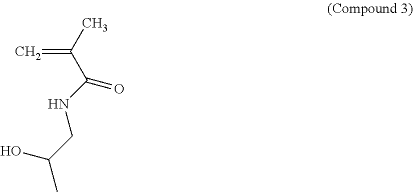
and a free radical initiator to provide a polymer of formula 8: 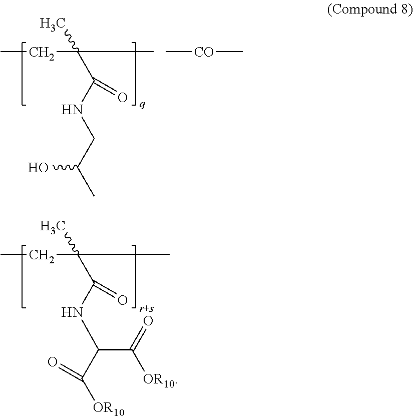
wherein:
‘co’ represents binding of the individual monomer units to form a random copolymer.
q, r, and s represent the molar percentage of the monomer units;
q+r+s=100;
q is ≧50 and ≦95;
r+s is ≧5 and ≦50.

9. The polymer of claim 3 of formula 10:

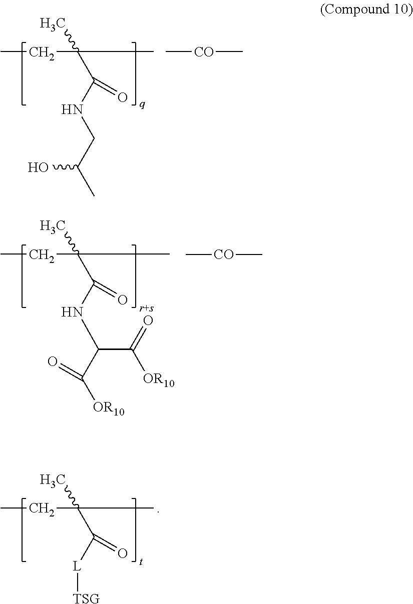

10. The polymer of any one of claims 2, 4, 7, and 9, wherein R₁₀ is methyl, ethyl, lower alkyl, or hydrogen.

11. A compound of formula 12: 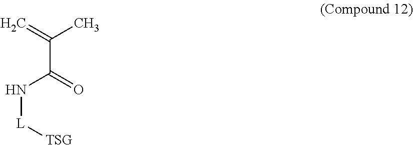
wherein L is a covalent bond, or an amino acid, or a peptide comprising 2-50 amino acids, or a straight or branched C₁-C₁₀ alkylene chain; or a combination of these components;
TSG is a tumor seeking group that is capable of targeting cancerous tumor selected from the group of a monoclonal antibody, an antibody fragment, a peptide, a protein, a steroid, a somatostatin analog, a lectin, a folic acid or its derivatives and analogs, vitamin B12, biotin, porphyrin, an essential fatty acid, a bioreductive molecule and a polyanionic polysaccharide.

12. A method of manufacture comprising contacting the compound of claim 11 with a compound of formula 3: 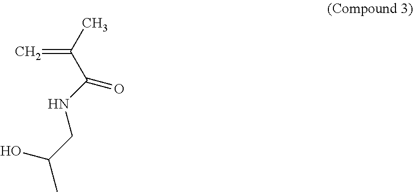
a compound of formula 4: 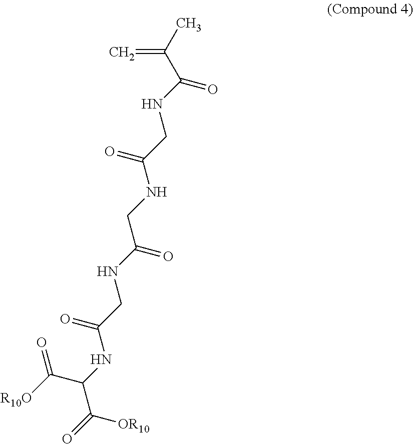
and a radical initiator to provide the polymer of claim 4 of formula 6.

13. A method of manufacture comprising contacting the compound of claim 11 with a compound of formula 3: 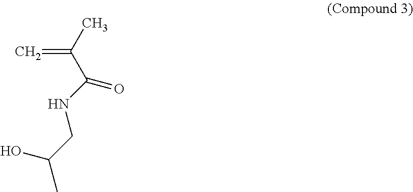
a compound of formula 7: 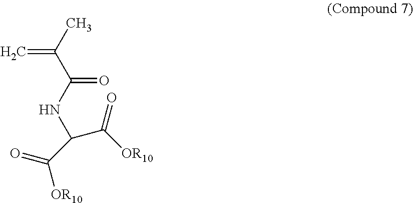
and a radical initiator to provide the polymer of claim 10 of formula 10.

14. The method of any one of claims 2, 8, and 13, wherein R₁₀ is methyl or ethyl.

15. A polymeric platinum complex comprising a polymer of formula 1a: 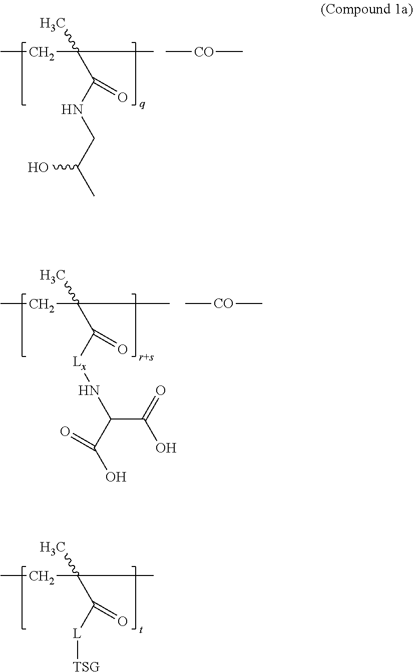
wherein:
‘co’ represents binding of the individual monomer units to form a random copolymer.
q, r+s, and t represent the molar percentage of the monomer units; q+r+s+t=100;
q is ≧50 and ≦95;
r+s is ≧5 and ≦50;
t is ≧0 and ≦30;
provided however that, t is 0 only when Lₓ is a bond;
TSG is a tumor seeking group that is capable of targeting cancerous tumor selected from the group of a monoclonal antibody, an antibody fragment, a peptide comprising 2-50 amino acids, a protein, a steroid, a somatostatin analog, a lectin, a folic acid or its derivatives and analogs, vitamin B12, biotin, porphyrin, an essential fatty acid, a bioreductive molecule and a polyanionic polysaccharide;
L and Lx are independently a covalent bond, or an amino acid, or a peptide comprising 2-50 amino acids, or a straight or branched C₁-C₁₀ alkylene chain; or a combination of these components;
and platinum;
or a salt of each of the above.

16. The polymeric platinum complex of claim 15 comprising a polymer of formula 6a: 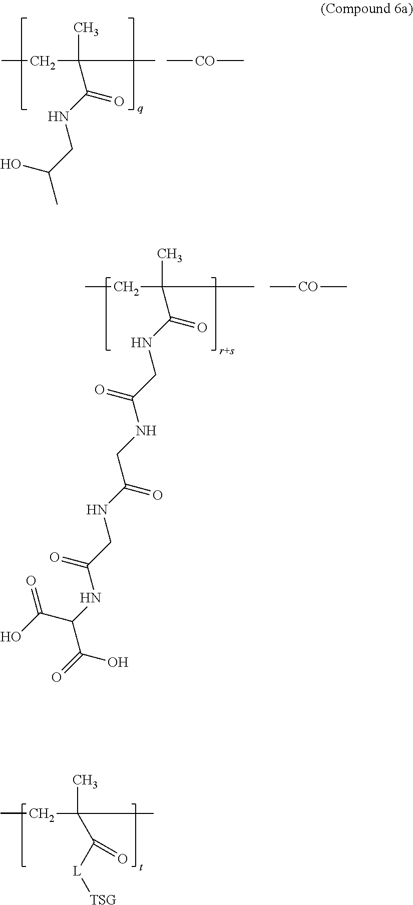
wherein
t is ≧1 and ≦30.

17. The polymeric platinum complex of claim 15 or 16 of formula 5: 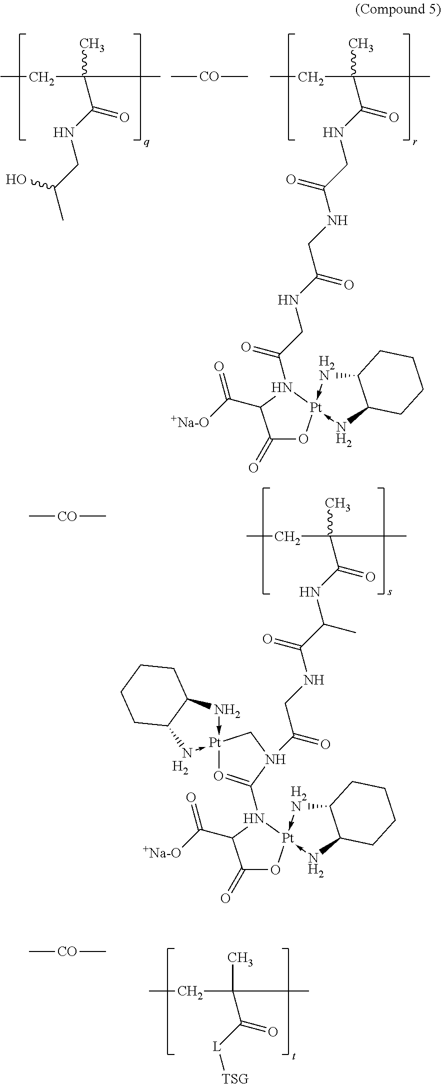
wherein
t is ≧0 and ≦30

18. The polymeric platinum complex of claim 15 of formula 9: 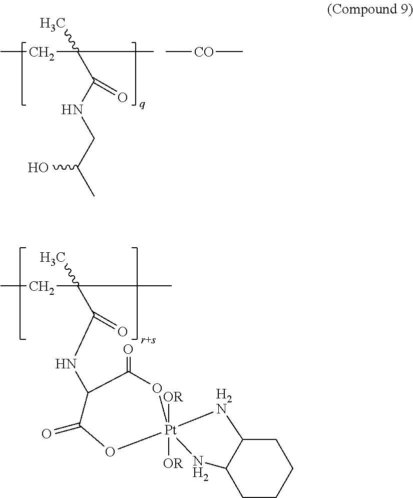
wherein:
the platinum is in the +4 oxidation state.

19. The polymeric platinum complex of claim 15 of formula 9a 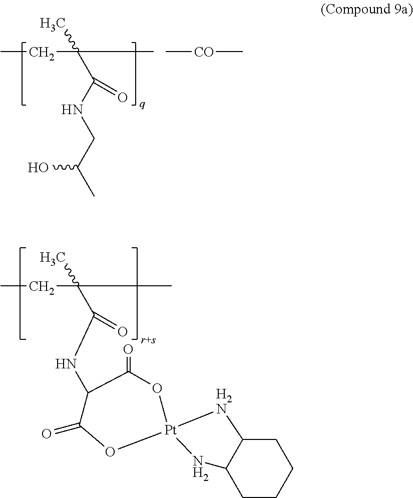
wherein:
the platinum is in the +2 oxidation state.

20. The polymeric platinum complex of claim 15 comprising a polymer of formula 10a: 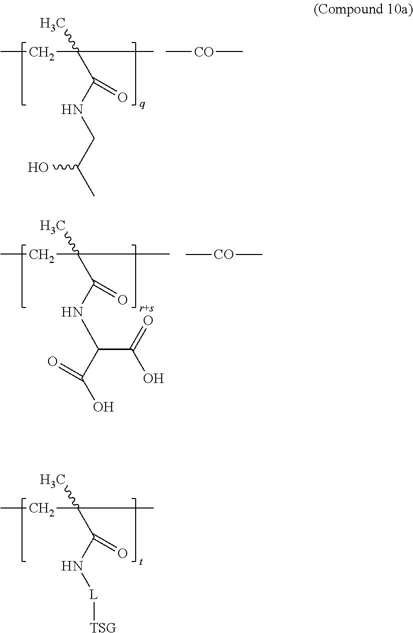
and platinum, wherein the platinum is in +2 oxidation state or in +4 oxidation state.

21. The polymeric platinum complex of claim 15, wherein at least about 80% of the complex is an N,O-amindomalonate platinum complex.

22. The polymeric platinum complex of claim 15, wherein at least about 80% of the complex is an O,O′-amindomalonate platinum complex.

23. The polymeric platinum complex of claim 15, wherein at least 5% of the platinum is in the +4 oxidation state.

24. A lyophilized unit dose pharmaceutical composition comprising the polymeric platinum complex of claim 15.

25. A pharmaceutical composition comprising the polymeric platinum complex of claim 15 and a pharmaceutically acceptable carrier, excipient, or diluent.

26. A method of treating cancer comprising administering a therapeutically effective amount of the polymeric platinum complex of claim 15.

27. The method of claim 26, further comprising administering another chemotherapeutic anticancer agent or radiation therapy.

28. The method of claim 26, wherein the cancer is a solid tumor cancer.

29. The method of claim 26, wherein the cancer is ovarian cancer, gastric cancer, lung cancer, or melanoma.

30. The method of claim 26, further comprising an anticancer agent selected from the group of paclitaxel, gemcitabine, 5-fluorouracil, 5-fluorouracil plus leucovorin, or vinorelbine.

31. The method of claim 26, wherein the compound is administered intravenously.

32. The method of claim 26, wherein the therapeutically effective amount is about 2 mg/kg to about 800 mg/kg.
